(19) United States
(12) Patent Application Publication
(10) Pub. No.: US 2006/0228405 A1
(43) Pub. Date: Oct. 12, 2006

(54) Phospholipid-based pharmaceutical formulations and methods for producing and using same

(75) Inventors: Edgar H. Ulm, San Diego, CA (US); Robert K. Mansfield, Carlsbad, CA (US); Gregg A. Timony, San Diego, CA (US); Marcus F. Boehm, San Diego, CA (US)

Correspondence Address: WILSON SONSINI GOODRICH & ROSATI, 650 PAGE MILL ROAD, PALO ALTO, CA, 94304-1050, US

(73) Assignee: Conforma Therapeutics Corporation

(21) Appl. No.: 11/279,088

(22) Filed: Apr. 7, 2006

Related U.S. Application Data
(60) Provisional application No. 60/669,591, filed on Apr. 7, 2005.

(51) Int. Cl. A61K 31/397; A61K 31/33; A61K 9/127

(52) U.S. Cl. 424/450; 514/183; 514/210.21

(57) ABSTRACT

Pharmaceutical formulations and methods of producing and using the same are described and claimed. The formulations are dispersions of phospholipids and one or more pharmacologically active compounds, pharmaceutically acceptable salts thereof, or prodrugs thereof. In specific embodiments, the pharmaceutically active compounds are ansamycins and the overall formulation is substantially devoid of medium and long chain triglycerides.

### CROSS-REFERENCE

This application claims the benefit of U.S. Provisional Application No. 60/669,591, filed Apr. 7, 2005, which is incorporated herein by reference in its entirety.

### FIELD OF INVENTION

The invention relates in general to pharmaceutical formulations and methods of producing and using the same; more particularly, the invention relates to phospholipid formulations of ansamycins, which are substantially devoid of medium and long chain triglycerides; more particularly, to phospholipid formulations of 17-allylamino-17-desmethyl-geldanamycin (17-AAG).

### BACKGROUND

The following description includes information that may be useful in understanding the present invention. It is not an admission that any of the information provided herein is prior art or relevant to the presently claimed inventions, or that any publication specifically or implicitly referenced is prior art.

Ansamycins are antibiotic molecules characterized by an “ansa” structure which comprises any one of benzoquinone, benzohydroquinone, naphthoquinone or naphthohydroquinone moieties bridged by a long chain. Geldanamycin (GDM) and its synthetic semi-synthetic analog 17-allylamino-17-desmethyl-geldanamycin (17-AAG) belong to the benzoquinone class of ansamycins. GDM, as first isolated from the microorganism Streptomyces hygroscopicus, was originally identified as a potent inhibitor of certain kinases, and was later shown to act by stimulating kinase degradation, specifically by targeting “molecular chaperones,” e.g., heat shock protein 90s (HSP90s). Subsequently, various other ansamycins have demonstrated more or less such activity, with 17-AAG being among the most promising and the subject of intensive clinical studies currently being conducted by the National Cancer Institute (NCI). See, e.g., Federal Register, 66(129): 35443-35444; Erlichman et al., Proc. AACR 2001, 42, abstract 4474.

HSP90s are ubiquitous chaperone proteins involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner J., TIBS, 1999, 24:136-141; Stepanova, L. et al., Genes Dev. 1996, 10:1491-502; Dai, K. et al., J. Biol. Chem. 1996, 271:22030-4). Studies further indicate that certain co-chaperones, e.g., HSP70, p60/Hop/Stil, Hip, Bagl, HSP40/Hdj2/Hsj1, immunophilins, p23, and p50, may assist HSP90 in its function (see, e.g., Caplan, A., Trends in Cell Biol., 1999, 9: 262-68).

Ansamycin antibiotics, e.g., herbimycin A (HA), geldanamycin, and 17-AAG, are thought to exert their anticancerous effects by tight binding to the N-terminus-binding pocket of HSP90 (Stebbins, C. et al., Cell, 1997, 89:239-250). This pocket is highly conserved and has weak homology to the ATP-binding site of DNA gyrase (Stebbins, C. et al., supra; Grenert, J. P. et al., J. Biol. Chem. 1997, 272:23843-50). Further, ATP and ADP have both been shown to bind this pocket with low affinity and to have weak ATPase activity (Proromou, C. et al., Cell, 1997, 90: 65-75; Panaretou, B. et al., EMBO J., 1998, 17: 482936). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., Proc. Natl. Acad. Sci. USA 1999, 96:1297-302; Schulte, T. W. et al., J. Biol. Chem. 1995, 270:24585-8; Whitesell, L., et al., Proc. Natl. Acad. Sci. USA 1994, 91:8324-8328). Ansamycins have also been demonstrated to inhibit the ATP-dependent release of chaperone-associated protein substrates (Schneider, C., L. et al., Proc. Natl. Acad. Sci. USA, 1996, 93:14536-41; Sepp-Lorenzino et al., J. Biol. Chem. 1995, 270:16580-16587). In either event, the substrates are degraded by a ubiquitin-dependent process in the proteasome (Schneider, C., L., supra; Sepp-Lorenzino, L., et al., J. Biol. Chem., 1995, 270:16580-16587; Whitesell, L. et al., Proc. Natl. Acad. Sci. USA, 1994, 91: 8324-8328).

This substrate destabilization occurs in tumor and non-transformed cells alike and has been shown to be especially effective on a subset of signaling regulators, e.g., Raf (Schulte, T. W. et al., Biochem. Biophys. Res. Commun. 1997, 239:655-9; Schulte, T. W., et al., J. Biol. Chem. 1995, 270:24585-8), nuclear steroid receptors (Segnitz, B., and U. Gehring. J. Biol. Chem. 1997, 272:18694-18701; Smith, D. F. et al., Mol. Cell. Biol. 1995, 15:6804-12), v-src (Whitesell, L., et al., Proc. Natl. Acad. Sci. USA 1994, 91:8324-8328) and certain transmembrane tyrosine kinases (Sepp-Lorenzino, L. et al., J. Biol. Chem. 1995, 270:16580-16587) such as EGF receptor (EGFR), Her2/Neu (Hartmann, F. et al., Int. J. Cancer 1997, 70:221-9; Miller, P. et al., Cancer Res. 1994, 54:2724-2730; Mimnaugh, E. G. et al., J. Biol. Chem. 1996, 271:22796-801; Schnur, R. et al., J. Med. Chem. 1995, 38:3806-3812), CDK4, and mutant p53 (Erlichman et al., Proc. AACR 2001, 42, abstract 4474). The ansamycin-induced loss of these proteins leads to the selective disruption of certain regulatory pathways and results in growth arrest at specific phases of the cell cycle (Muise-Heimericks, R. C. et al., J. Biol. Chem. 1998, 273:29864-72), and apoptsosis, and/or differentiation of cells so treated (Vasilevskaya, A. et al., Cancer Res., 1999, 59:3935-40).

In addition to anti-cancer and antitumorigenic activity, HSP90 inhibitors have also been implicated in a wide variety of other utilities, including use as anti-inflammation agents, anti-infectious disease agents, agents for treating autoimmunity, agents for treating stroke, ischemia, cardiac disorders and agents useful in promoting nerve regeneration (See, e.g., Rosen et al., PCT Publication No. WO 02/09696 (PCT/USO1/23640); Degranco et al., WO 99/51223 (PCT/US99/07242); Gold, U.S. Pat. No. 6,210,974 B1; DeFranco et al., U.S. Pat. No. 6,174,875). Overlapping somewhat with the above, there are reports in the literature that fibrogenetic disorders, including but not limited to scleroderma, polymyositis, systemic lupus, rheumatoid arthritis, liver cirrhosis, keloid formation, interstitial nephritis, and pulmonary fibrosis, also may be treatable. (Strehlow, WO 02/02123 (PCT/US01/20578)). Still further HSP90 modulation, modulators and uses thereof are reported in International Application Nos. PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, PCT/US03/10533, PCT/US03/02686, and U.S. Provisional Application Nos. 60/293,246, 60/371,668, 60/331,893, 60/335,391, 60/128,593, 60/337,919, 60/340,762, and 60/359,484.

Ansamycins thus hold great promise for the treatment and/or prevention of many types of disorders. However, like many other lipophilic drugs, they are difficult to prepare for pharmaceutical applications, especially injectable intravenous formulations. To date, attempts have been made to use lipid vesicles and oil-in-water type emulsions, but these have thus far included complicated processing steps, harsh or clinically unacceptable solvent use, formulation instability, and/or irritation at the site of injection. See generally Vemuri, S. and Rhodes, C. T., Preparation and characterization of liposomes as therapeutic delivery systems: a review, Pharmaceutica Acta Helvetiae 1995, 70, pp. 95-111; see also PCT/US99/30631, published Jun. 29, 2000 as WO 00/37050.

A need therefore exists for alternative formulation methods and products that can ameliorate or negate one or more of these deficiencies, and the present invention satisfies that need.

### SUMMARY OF THE INVENTION

The invention features pharmaceutical formulations and methods of producing and using the same. The formulations are dispersions comprised of complexes of phospholipids and one or more pharmaceutically active compounds, or a polymorph, solvate, ester, tautomer, enantiomer, pharmaceutically acceptable salt, or a prodrug thereof.

In many of the embodiments, the pharmaceutically active compounds are ansamycins and the overall formulation is substantially devoid of medium and long chain triglycerides. The formulations can be filter-sterilized, lyophilized and/or frozen and, depending on the specific lipophilicity/hydrophobicity of the compound(s) used, offer the advantage of providing for higher concentrations of lipophilic compound per aqueous physiological unit volume than would otherwise be possible in noncomplexed form using known methods such as emulsification. Dilution ability is also enhanced by the formulations and methods of the invention, as is subject tolerability at the site of intravenous injection when used for such. Without being bound by theory, Applicants believe the latter to be due to the greater physiological compatibility of the phospholipids and relatively large proportions thereof used in the formulations of the invention.

A first aspect of the invention relates to pharmaceutical formulations. Each of these pharmaceutical formulations contains a pharmaceutically effective amount of an ansamycin, or a polymorph, solvate, ester, tautomer, enantiomer, pharmaceutically acceptable salt or a prodrug thereof, and a pharmaceutically acceptable phospholipid to form aqueous dispersible particles, wherein the formulation is substantially devoid of medium and long chain triglycerides, and the phospholipid is present at a concentration of at least 5% w/w of said formulation. In some embodiments, the medium and long chain triglycerides are present at a combined concentration of about 1% w/v or less.

Any pharmaceutically active ansamycins maybe used in the pharmaceutical formulations of the invention. In some embodiments, the ansamycin is selected from the following compounds:

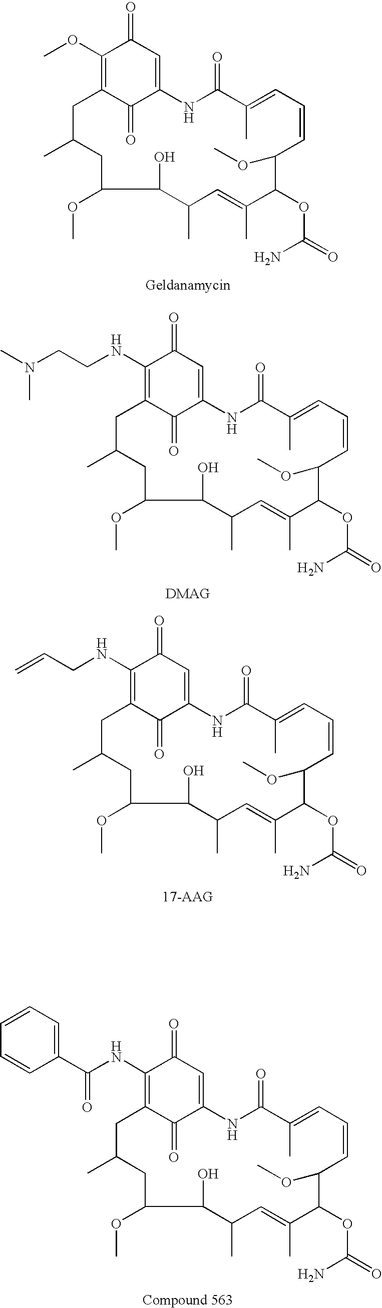

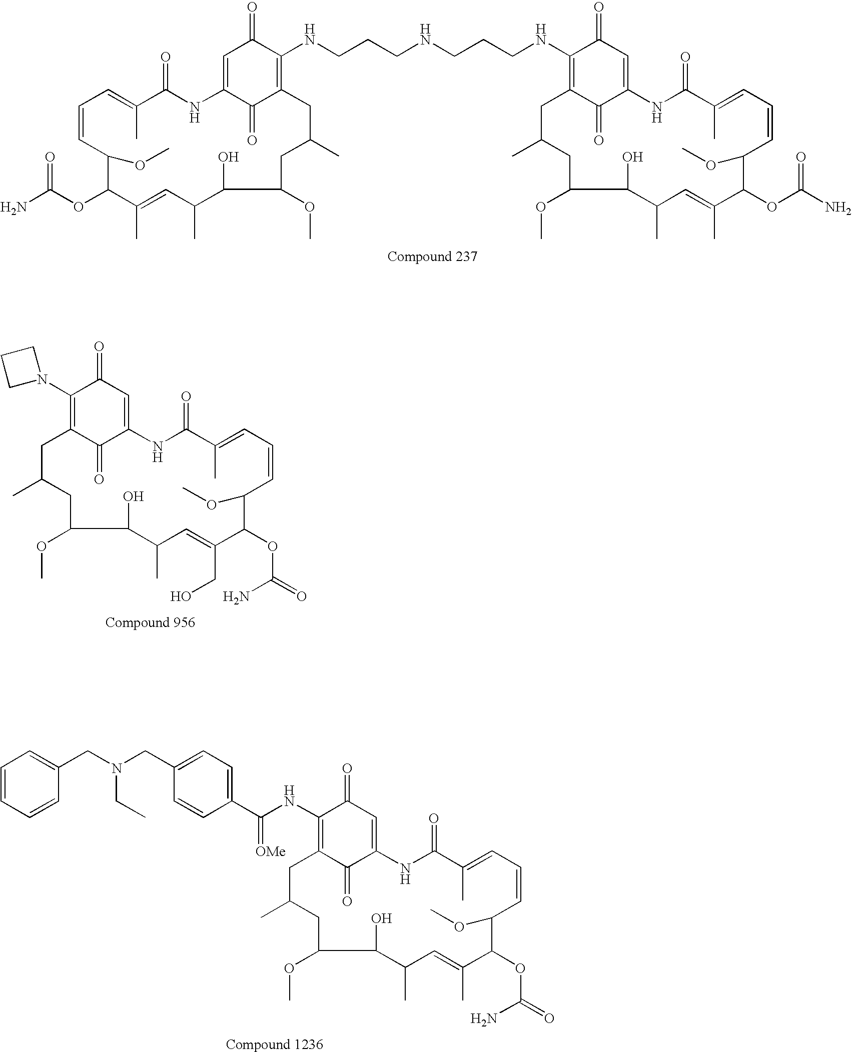

In some embodiments, the ansamycin is 17-AAG. In some other embodiments, the 17-AAG is in the form of a hydrochloride salt or a phosphate salt. In some other embodiments, the 17-AAG is the high melt form or polymorph, the low melt form, the amorphous form, or any combinations of the above forms. In some embodiments, the low melt form of 17-AAG is characterized by DSC melting temperatures below 175° C. and by an X-ray powder diffraction pattern comprising peaks located at 5.85 degree, 4.35 degree and 7.90 degree two-theta angles. In some other embodiments, the low melt form of 17-AAG is characterized by a DSC melting temperature of about 156° C. and by an X-ray powder diffraction pattern comprising peaks located at 5.85 degree, 4.35 degree and 7.90 degree two-theta angles. In yet other embodiments, the low melt polymorph of 17-AAG characterized by a DSC melting temperature of about 172° C.

Additionally, the concentration of the ansamycin, or a polymorph, solvate, ester, tautomer, enantiomer, pharmaceutically acceptable salt, or prodrug thereof, in the pharmaceutical formulations of the invention may be at a concentration of about 0.5 mg/mL, about 5.0 mg/mL, about 50 mg/mL, or more.

The phospholipids in some embodiments of the pharmaceutical formulations of the invention may include one or more members selected from phosphatidylcholine, phosphatidalserine, phosphatidylinositol, phosphatidalethanolamine, and Phospholipon 90G. In some particular embodiments, the phospholipids include Phospholipon 90G. The particle size of the aqueous dispersible particles may be reduced using one or more of sonication, high shear homogenization, microfluidization, and extrusion through controlled pore size filters. The particle size of the aqueous dispersible particles is about 200 nm or less. In some embodiments, the particle size is between about 100 and 200 nm. In other embodiments, the particle size is between about 50 nm and 200 nm, and in other embodiments, the particle size is colloidal. Further, some embodiments of the pharmaceutical formulations of the invention include one or more excipients which may serve as one or more of cryoprotectant, tonicity modifier and bulking agent.

A second aspect of the invention relates to methods of preparing ansamycin pharmaceutical formulations. The preparative method includes the following steps:

(a) forming dispersion particles comprising

an ansamycin, or a polymorph, solvate, ester, tautomer, enantiomer, pharmaceutically acceptable salt or prodrug thereof; and
a pharmaceutically acceptable phospholipid;
   (b) optionally reducing the size of said dispersion particles;
   (c) optionally freezing the product of step (a) or (b);
   (d) optionally thawing the product of step (c);
   (e) optionally lyophilizing the product of any of steps (a)-(d); and
   (f) optionally rehydrating the product of step (e); and

wherein said formulation is substantially devoid of medium and long chain triglycerides.

The method of the invention, in some embodiments, may further include adding one or more excipients which serve as one or more of cryoprotectant, tonicity modifier and bulking agent.

The method is for the preparation of pharmaceutical formulations of ansamycin, in particular, geldanamycin, 17-AAG, DMAG, Compound 563, Compound 237, Compound 956, Compound 1236, or combinations thereof. In some embodiments, the method is for the preparation of pharmaceutical formulations of 17-AAG, geldanamycin or DMAG. In particular embodiments, the method is for the preparation of pharmaceutical formulations of 17-AAG. In other embodiments, the method is for the preparation of pharmaceutical formulations of the high melt, low melt, amorphous forms, or any combinations thereof, of 17-AAG. In some particular embodiments, the method is for the preparation of pharmaceutical formulations of a low melt form of 17-AAG.

The concentration of the ansamycin, pharmaceutically acceptable salt thereof, or prodrug thereof, in the pharmaceutical formulation prepared by the method of the invention is at least about 0.5 mg/mL in some embodiments, is at least about 5.0 mg/mL in other embodiments and is at least about 50 mg/mL or more in yet other embodiments.

The phospholipids used in the methods of the invention include phosphatidylcholine, phosphatidylserine, phosphatidylinositol, phosphatidylethanolamine, Phospholipon 90G, or any combination thereof. In some embodiments, the phospholipids used include phosphatidylcholine, Phospholipon 90G, Phospholipon 90G, or any combination thereof. In other embodiments, the phospholipids used include phosphatidylcholine, phosphatidylethanolamine, Phospholipon 90G, or any combination thereof. In some particular embodiments, the phospholipids used include Phospholipon 90G.

The method of preparing the pharmaceutical formulation may include a step of reducing the particle size of the dispersion particles. In some embodiments, the particle size reduction is accomplished using one or more of sonication, high shear homogenization, microfluidization, and extrusion through controlled pore size filters. In some embodiments, the reduction is accomplished using high shear homogenization and/or microfluidization. In other embodiments, the reduction is accomplished using high shear homogenization and/or extrusion through controlled pore size filters. The method, in some embodiments, produces dispersion particles having particle sizes that are colloidal, that are between about 50 and 200 nm, that are between about 100 and 200 nm, or that are about 200 nm or less. In some embodiments, the particle sizes are between about 100 and 200 nm. In other embodiments, the particle sizes are about 200 nm or less. In yet other embodiments, the particles sizes are colloidal.

A third aspect of the invention is related to methods of treating or preventing a disorder in a mammal, by administering to a mammal a pharmaceutically effective amount of any of the pharmaceutical formulations which is the first aspect of the invention or a pharmaceutical formulation made by any of the preparative methods.

The treatment method may be used to treat ischemia, proliferative disorders, infections, neurological disorders, tumors, leukemias, chronic lymphocytic leukemia, neoplasms, cancers, carcinomas, acquired immunodeficiency syndrome, and malignant diseases. Among the proliferative disorders, against which the method is applicable are tumors, inflammatory diseases, fungal infection, yeast infection, and viral infection.

In some embodiments of the treatment method of the invention, the ansamycin, or a polymorph, solvate, ester, tautomer, enantiomer, pharmaceutically acceptable salt or a prodrug thereof, is administered at a concentration of about 1-1.5% (w/w) in the pharmaceutical formulation, or at a concentration of between about 0.5 and 50 mg/ml.

In some embodiments of the treatment method, the ansamycin in the pharmaceutical formulations is selected from geldanamycin, DMAG, 17-AAG, Compound 563, Compound 237, Compound 956, and Compound 1236. In some embodiments, the ansamycins is 17-AAG. In other embodiments, the 17-AAG is selected from a high melt, a low melt, an amorphous form of 17-AAG, or any combinations thereof. In yet other embodiments, the ansamycin comprises a low melt form of 17-AAG.

A fourth aspect of the invention is the use of the phospholipid formulations of the invention in the manufacture of a medicament.

Yet another aspect of the invention is the use of the phospholipid formulations of the invention in the manufacture of medicaments for the therapeutic and prophylactic treatment of HSP90 mediated diseases and conditions discussed above.

It should be understood that any of the above described aspects and embodiments of the invention can be combined in anyway where practical; those of ordinary skill in the art will appreciate the ways the various embodiments may be combined usefully within the spirit of the invention.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the X-ray powder diffraction pattern of the high melt form of 17-AAG showing peaks at 7.40, 6.08 and 11.84 two-theta angles.

FIG. 2 shows the X-ray powder diffraction pattern of the low melt form of 17-AAG showing peaks at 5.85, 4.35 and 7.90 two-theta angles.

FIG. 3 shows a differential scanning calorimetry (DSC) scan of the high melt form of 17-AAG.

FIG. 4 shows a DSC scan of the low melt form of 17-AAG.

FIG. 5 shows the intrinsic dissolution rate (mg/cm²) of low melt and high melt 17-AAG versus time (min) in ethanol.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The invention features phospholipid-based pharmaceutical formulations of ansamycins and methods of producing and using the same. Applicants have observed that water-soluble or slightly water-soluble ansamycins or water-soluble salts of water-insoluble ansamycins can be formulated into dispersions of pharmaceutically acceptable phospholipids. Applicants further observed that different polymorphic forms of crystalline ansamycins have different dissolution characteristics, e.g., 17-AAG has low melt forms which exhibit significantly higher dissolution rates than the high melt forms. Taking advantage of these properties, Applicants have devised formulations for water-insoluble drugs, e.g., ansamycins, that are suitable for administration to a patient. The preparation of such a formulation is relatively simple, typically utilizes clinically acceptable reagents, and results in a product that affords storage stability.

The present invention differ from the emulsion formulations described in PCT/US03/10533 in that the present formulations contain lower levels of medium chain triglycerides (MCT) and long chain triglycerides. MCT can lead to metabolic formation of octanoate, which can lead to central nervous system effects such as somnolence, nausea, drowsiness and changes in EEG. See Cotter et al., Am. J. Clin. Nutr. 1090 50:794-800; Miles et al., Journal of Parenteral and Enteral Nutrition 1991 15:37-41; Traul et al., Food Chem. Toxicol. 2000 38:79-98. Additionally, Applicants' presently claimed formulations are well tolerated during intravenous administration.

While the invention is illustrated herein using an ansamycin, 17-AAG, it should be understood that the novel method of drug formulation described herein applies to other lipophilic, low water solubility drugs. It should be also understood that the method further applies to many other ansamycins including, but are not limited to, those exemplified in Examples 1-12 of the EXAMPLE section, such as geldanamycin, 17-N,N-dimethylaminoethylaminogeldanamycin (DMAG), and 17-AAG. It further should be understood that the novel method of drug formulation described herein applies to both the high melt and low melt forms of 17-AAG. Yet further, the formulation further applies to the polymorphs, tautomers, enantiomers, pharmaceutically acceptable salts and prodrugs of the disclosed compounds.

I. Definitions

The following claim terms have the following meanings, and claim terms not specifically appearing below have their common customary meaning as used in the art:

The term “prodrug” or “pharmaceutically acceptable prodrug” is a pharmaceutically active drug covalently bonded to a carrier wherein release of the pharmaceutically active drug occurs in vivo when the prodrug is administered to a mammalian subject. Prodrugs of the compounds of the present invention are prepared by modifying functional groups present in the compounds in such a way that the modified groups are cleaved, either in routine manipulation or in vivo, to yield the desired compound. Prodrugs include compounds wherein hydroxy, amine, or sulfhydryl groups are bonded to any group that, when administered to a mammalian subject, is cleaved to form a free hydroxyl, amino, or sulfhydryl group, respectively. Examples of prodrugs include, but are not limited to, acetate, formate, or benzoate derivatives of alcohol or amine functional groups in the compounds of the present invention; phosphate esters, dimethylglycine esters, aminoalkylbenzyl esters, aminoalkyl esters or carboxyalkyl esters of alcohol or phenol functional groups in the compounds of the present invention; or the like. Prodrugs can impart multiple advantages for drug delivery, e.g., as explained in REMINGTON'S PHARMACEUTICAL SCIENCES, 20th Edition, Ch. 47, pp. 913-914.

“Pharmaceutically acceptable salts” include those derived from pharmaceutically acceptable inorganic and organic acids and bases. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-d-gluconic acid and the like. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N-(C₁-C₄ alkyl)₄+salts, and the like. Illustrative examples of some of these include sodium hydroxide, potassium hydroxide, choline hydroxide, sodium carbonate, and the like. Where the claims recite “a compound (e.g., compound ‘x’) or pharmaceutically acceptable salt thereof,” and only the compound is displayed, those claims are to be interpreted as embracing, in the alternative or conjunctive, a pharmaceutically acceptable salt or salts of such compound.

A “pharmaceutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate, half-life and maximum tolerated dose (MTD) have yet to be determined but one of ordinary skill in the art can determine these using standard procedures.

Some of the compounds described herein may contain one or more chiral centers and therefore may exist in enantiomeric and diastereomeric forms. The scope of the present invention is intended to cover all isomers per se, as well as mixtures of cis and trans isomers, mixtures of diastereomers and racemic mixtures of enantiomers (optical isomers) as well. Further, it is possible using well known techniques to separate the various forms, and some embodiments of the invention may feature purified or enriched species of a given enantiomer or diastereomer. In addition, some of the compounds of the present invention may exist as tautomers, which are isomers that differ by the placement of a proton and the corresponding location of a double bond. The scope of the present invention is intended to cover all tautomeric forms. Further, the compounds described herein may exist as solvates, which refers to the combination of said compounds, or the ions of said compounds, with one or more solvent molecules. The scope of the present invention is intended to cover all solvated forms of the compounds described herein.

The terms “dispersion”, “colloid” and “emulsion” have meanings in the art consistent with REMINGTON'S THE SCIENCE AND PRACTICE OF PHARMACY, 20th Edition, Gennaro, A. R. Ed., (2000) and denote multiphasic systems comprised of two or more ingredients that are not completely miscible in one another. Dispersions may be classified into different groups based on the size of the dispersed particles. Colloidal dispersions are characterized by dispersed particles in the range of approximately 1 nm to 0.5 μm. Coarse dispersions are characterized by particle sizes exceeding 0.5 μm, and include suspensions and emulsions. For the most part, the different types of dispersions can be detected by light-scattering and/or microscopic techniques, including, e.g. electron microscopy.

“Lyophilization” is the removal or substantial removal of liquid from a sample, e.g., by sublimation. Solvent/aqueous phase removal may be accomplished using any procedure but is generally accomplished under reduced pressure, i.e., vacuum, at any reasonable temperature and pressure, including at room temperature with a stream of nitrogen, as long as suitable to preserve the functional integrity of the pharmaceutically active drug. The terms “lyophilizing” and “lyophilized” do not necessarily imply 100% elimination of solvent and/or solution, and may entail lesser percentages of removal. Substantial removal is typically about 95% removal.

An “inert atmospheric condition” is one that is relatively less reactive than the air of standard atmospheric conditions. The use of pure or substantially pure nitrogen gas is one example of such an inert atmospheric condition. Persons of ordinary skill in the art are familiar with others.

The term “hydrating” or “rehydrating” means adding an aqueous solution, e.g., water or a physiologically compatible buffer such as Hanks's solution, Ringer's solution, physiological saline buffer, or 5% dextrose in water.

A “physiologically acceptable carrier” refers to a carrier or diluent that does not cause significant irritation to an organism and does not abrogate the biological activity and properties of the administered compound.

The term “excipient” refers to a non-toxic pharmaceutically acceptable substance added to a pharmacological composition to facilitate the processing, administration, and physical characteristic of a compound. Examples of excipients may include, but are not limited to, calcium carbonate, calcium phosphate, various sugars including mannitol, sucrose, and/or dextrose, and types of starch, cellulose derivatives, gelatin, various buffering agents such as sodium acetate, phosphate, lactate, tartrate and/or maleate, amino acids, sugar acids (e.g., glucocoronate and/or gluconate), and thixotropic agents such as polyethylene glycol, polyvinyl pyrrolidone and/or poloxamers (co-polymers).

The term “stabilizer” can be synonymous with “bulking agent” or “freeze-drying agent” and vice versa, although need not be. “Bulking agents” are a type of excipient that generally provide mechanical support for a lyophile formulation by allowing the dry formulation matrix to maintain its conformation. Typically, the bulking agents are sugars. Sugars as used herein include but are not limited to monosaccharides, disaccharides, oligosaccharides and polysaccharides. Specific examples include but are not limited to fructose, glucose, mannose, trehalose, sorbose, xylose, maltose, lactose, sucrose, dextrose, and dextran. Sugar also includes sugar alcohols, such as mannitol, sorbitol, inositol, dulcitol, xylitol and arabitol. Mixtures of sugars may also be used in accordance with this invention. Various bulking agents, e.g., glycerol, sugars, sugar alcohols, and mono- and di-saccharides may also serve the function of isotonizing agents. Bulking agents for use with the invention are limited only by chemico-physical considerations, such as solubility, ability to preserve the droplet size and emulsion integrity during freezing, drying, storage and rehydration and lack of reactivity with the active drug/compound, and limited as well by route of administration. Generally, the bulking agents be chemically inert to drug(s) and have no or extremely limited detrimental side effects or toxicity under the conditions of use. In addition to bulking agent carriers, other carriers that may or may not serve the purpose of bulking agents include, e.g., adjuvants, excipients, and diluents as well known and readily available in the art. An exemplary bulking agent for the invention is sucrose. Without being bound by theory, sucrose is thought to form an amorphous glass upon freezing and subsequent lyophilization, allowing for potential stability enhancement of the formulation by forming a dispersion wherein the drug-phospholipid complex is contained in a rigid glass. Stability may also be enhanced by virtue of the sugar acting as a replacement for the water lost upon lyophilization. The sugar molecules, rather than the water molecules, become bonded to the interfacial phospholipid through hydrogen bonds. Other bulking agents which possess these characteristics and which may be substituted include but are not limited to polyvinylpyrrolidone (PVP) and mannitol.

The term “ansamycin” is a broad term which characterizes compounds having an “ansa” structure which comprises any one of benzoquinone, benzohydroquinone, naphthoquinone or naphthohydroquinone moities bridged by a long aliphatic chain. Compounds of the naphthoquinone or naphthohydroquinone class are exemplified by the clinically important agents rifampicin and rifamycin, respectively. Compounds of the benzoquinone class are exemplified by geldanamycin (including its synthetic derivatives 17-allylamino-17-demethoxygeldanamycin (17-AAG), 17-N,N-dimethylaminoethylamino-17-demethoxygeldanamycin (DMAG)), dihydrogeldanamycin and herbamycin. The benzohydroquinone class is exemplified by macbecin. The term “ansamycins” as used herein can also embrace pharmaceutically acceptable salts of ansamycins, as well as ansamycin prodrugs, which upon administration to an individual metabolize into more or less pharmacological active compounds. Prodrugs are typically employed to enhance one or more of solubility, delivery and/or biological presence and persistence of a pharmacological compound in a subject patient.

The term “phospholipid” includes any lipid containing phosphoric acid as mono- or di-ester. The phospholipids of the invention may be synthetic, natural, or semi-synthetic and may, although not necessarily, share identity with known cellular membrane phospholipids such as phosphoglycerides and sphingomyelin.

The term “phosphoglyceride” as used herein, refers to a compound derived from glycerol, a three-carbon alcohol, and possessing a glycerol backbone esterified to two fatty acid chains via two glycerol hydroxyl groups, and esterified to phosphoric acid via the remaining hydroxyl group to form an intermediate, phosphatidate. The fatty acid chains typically contain between 14 and 24 carbon atoms, with 16 and 18 being the most common. The chains may be either saturated or unsaturated. The phosphate group itself is then esterified to the hydroxyl group of one of several different alcohols, with the most common being serine, ethanolamine, choline, glycerol, and inositol. Exemplary phosphoglycerides include, but are not limited to, phosphatidylcholine (PC), phosphatidylserine (PS), phosphatidylinositol (PI), phosphatidylethanolamine (PE). Sphingomyelin is derived from sphingosine, an amino alcohol that contains a long, unsaturated hydrocarbon chain. In sphingomyelin, the amino group of the sphingosine backbone is linked to a fatty acid by an amide bond. In addition, the primary hydroxyl group of sphingosine is esterified to phosphoryl choline. See, e.g., Stryer, BIOCHEMISTRY, Second Edition, pp. 206-211 (1981).

Additionally, phosphoglycerides also include lecithins. “Lecithins” are naturally occurring mixtures of diglycerides of stearic, palmitic, and oleic acids, linked to the choline ester of phosphoric acid. Preferred phospholipids for use with the invention are soya lecithin, e.g., Phospholipon 90G as supplied by American Lecithen Company (Oxford, Conn., USA). Other commercial sources and methods of preparation are known to the skilled artisan. For example, TWEEN® 80 (polyoxyethylene sorbitan monooleate) and Poloxamer 188 are other commercial reagents envisioned to work.

The phospholipids of the invention are typically present in concentrations of about 0.5-20% w/v based on the amount of the water and/or other components into which the surfactant is dissolved. Generally, the phospholipid is present in a concentration of about 0.5-10% w/v, typically about 1-8% w/v.

To prevent or minimize oxidative degradation or lipid peroxidation, antioxidants, e.g., alpha-tocopherol and butylated hydroxytoluene, may be included in addition to, or as an alternative to, oxygen deprivation (e.g., formulation in the presence of inert gases such as nitrogen and argon, and/or the use of light resistant containers).

The term “trigylceride” as used herein refers to a triester of glycerol (HO—CH(CH₂OH)₂). The three ester groups may be identical, two of the three may be the same, with the third being different or all three may be different

The term “short chain triglyceride” as used herein, refers to a triglyceride comprising ester groups containing less than 8 linear carbon atoms.

The term “medium chain triglyceride” as used herein, refers to a triglyceride comprising ester groups containing 8 to 12 linear carbon atoms.

The term “long chain triglyceride” as used herein, refers to a triglyceride comprising ester groups containing greater than 12 linear carbon atoms.

The term “about” means including and exceeding up to 20% the specific endpoint(s) designated. Thus the range is broadened.

The term “optionally” denotes that the step or component following the term may, but need not be, a part of the method or formulation.

The term “substantially devoid of” as pertains to medium and long chain triglycerides means that these items singularly comprise 5% w/v (collectively 10% w/v) or less of the entire formulation. Thus any range of from about 0 to 5% medium or long chain triglyceride species constitutes “substantially devoid.”

II. Preparation of the Formulations

A. Preparation of Ansamycins

Ansamycins according to this invention may be synthetic, naturally-occurring, or a combination of the two, i.e., “semi-synthetic,” and may include dimers and conjugated variant and prodrug forms. Some exemplary benzoquinone ansamycins useful in the various embodiments of the invention and their methods of preparation include but are not limited to those described, e.g., in U.S. Pat. No. 3,595,955 (describing the preparation of geldanamycin), No. 4,261,989, No. 5,387,584, and No. 5,932,566 and those described in the “EXAMPLE” section (Examples 1-12), below. The biochemical purification of the geldanamycin derivative, 4,5-dihydrogeldanamycin and its hydroquinone from cultures of Streptomyces hygroscopicus (ATCC 55256) are described in Cullen et. al. as WO 93/14215; an alternative method of synthesis for 4,5-dihydrogeldanamycin by catalytic hydrogenation of geldanamycin is also known. See e.g., “Progress in the Chemistry of Organic Natural Products,” Chemistry of the Ansamycin Antibiotics, 1976 33:278. Other ansamycins that can be used in connection with various embodiments of the invention are described in the literature cited in the “Background” section above. In addition, geldanamycin and DMAG are also commercially available, e.g., from CN Biosciences, an Affiliate of Merck KGaA, Darmstadt, Germany, headquartered in San Diego, Calif., USA (cat. no. 345805) and EMD/Calbiochem an Affiliate of Merck KGaA, Darmstadt, Germany, respectively.

17-AAG may be prepared from geldanamycin via reaction with allyamine in dry THF under a nitrogen atmosphere. The crude product may be purified by slurrying in H₂O:EtOH (90:10), and the washed crystals obtained have a melting point of 206-212° C. by capillary melting point technique. A second product of 17-AAG can be obtained by dissolving and recrystallizing the crude product from 2-propyl alcohol (isopropanol). This second 17-AAG product has a melting point between 147-153° C. by capillary melting point technique. The two 17-AAG products are designated as the “high melt form or polymorph” and “low melt form.” The stability of the low melt form may be tested by slurring the crystals in the solvent (H₂O:EtOH (90:10)) from which the high melt form was purified; no conversion to the high melt form was observed. See Examples 1-2 for details of the preparation of the two polymorphic forms of 17-AAG. In addition to the high melt and low melt forms, it is well known that 17-AAG has an amorphous form.

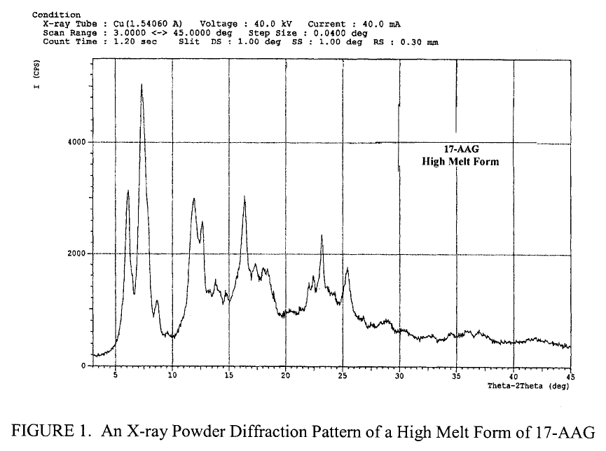

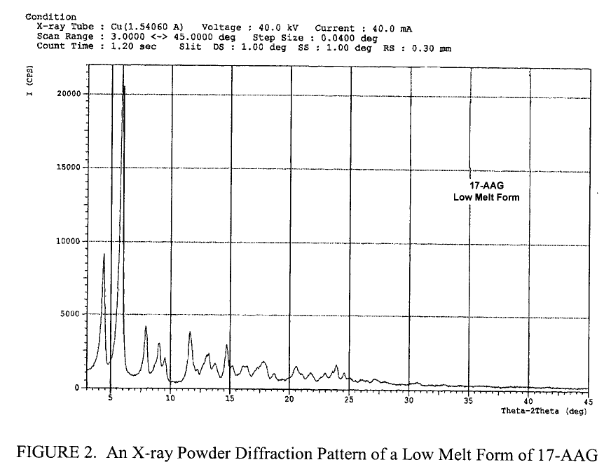

The presence of different polymorphic forms may be assessed by X-ray powder diffraction and by differential scanning calorimetry (DSC). Distinctively different X-ray powder diffraction patterns are indicative that the materials are of different crystalline structures. FIG. 1 shows the X-ray powder diffraction pattern of the high melt form which includes peaks at 7.40 degree, 6.08 degree and 11.84 degree two-theta angles. FIG. 2 shows the X-ray powder diffraction pattern of the low melt 17-AAG which includes peaks at 5.85 degree, 4.35 degree and 7.90 degree two-theta angles. Since the X-ray powder diffraction patterns are distinctly different, the high melt and low melt 17-AAG contain different crystalline forms of 17-AAG.

The peak locations and intensities of the X-ray powder diffraction patterns for the high melt form and low melt form of 17-AAG are summarized in Table 1 and Table 2, respectively.

TABLE 1
X-Ray Powder Diffraction Pattern of A High Melt 17-AAG
17-AAG High Melt Form
# Strongest 3 peaks

| | | | | | | | Integrated |
|---|---|---|---|---|---|---|---|
| | peak | 2Theta | d | | FWHM | Intensity | Int |
| no. | no. | (deg) | (A) | I/I1 | (deg) | (Counts) | (Counts) |
| 1 | 2 | 7.4042 | 11.92989 | 100 | 0.88940 | 3462 | 77678 |
| 2 | 1 | 6.0824 | 14.51916 | 57 | 0.73690 | 1964 | 40942 |
| 3 | 5 | 11.8400 | 7.46851 | 52 | 0.81900 | 1810 | 32565 |

# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 6.0824 | 14.51916 | 57 | 0.73690 | 1964 | 40942 |
| 2 | 7.4042 | 11.92989 | 100 | 0.88940 | 3462 | 77678 |
| 3 | 8.6000 | 10.27358 | 14 | 0.63020 | 472 | 9907 |
| 4 | 10.7200 | 8.24615 | 4 | 0.34660 | 125 | 1866 |
| 5 | 11.8400 | 7.46851 | 52 | 0.81900 | 1810 | 32565 |
| 6 | 12.4800 | 7.08691 | 40 | 0.91960 | 1386 | 36608 |
| 7 | 13.8800 | 6.37508 | 16 | 0.00000 | 546 | 0 |
| 8 | 14.7200 | 6.01312 | 11 | 0.00000 | 366 | 0 |
| 9 | 16.3120 | 5.42966 | 45 | 0.88790 | 1566 | 50640 |
| 10 | 17.3200 | 5.11587 | 22 | 0.00000 | 746 | 0 |
| 11 | 18.1600 | 4.88108 | 21 | 1.36660 | 711 | 26508 |
| 12 | 20.4400 | 4.34147 | 3 | 1.38660 | 110 | 4924 |
| 13 | 22.2400 | 3.99400 | 15 | 0.93120 | 524 | 11702 |
| 14 | 23.1340 | 3.84163 | 28 | 0.82570 | 961 | 22215 |
| 15 | 24.1200 | 3.68678 | 12 | 0.00000 | 400 | 0 |
| 16 | 25.3229 | 3.51431 | 21 | 0.86220 | 717 | 20392 |
| 17 | 26.6400 | 3.34347 | 3 | 0.66660 | 116 | 3106 |
| 18 | 28.7575 | 3.10191 | 4 | 1.19500 | 153 | 6842 |
| 19 | 36.0400 | 2.49007 | 4 | 1.77600 | 143 | 7021 |
| 20 | 36.9200 | 2.43271 | 4 | 1.60000 | 130 | 4256 |

TABLE 2
X-Ray Powder Diffraction Pattern of A Low Melt 17-AAG
17-AAG
Low Melt Form
# Strongest 3 peaks

| | | | | | | | Integrated |
|---|---|---|---|---|---|---|---|
| | peak | 2Theta | d | | FWHM | Intensity | Int |
| no. | no. | (deg) | (A) | I/I1 | (deg) | (Counts) | (Counts) |
| 1 | 2 | 5.8457 | 15.10652 | 100 | 0.40550 | 14036 | 168505 |
| 2 | 1 | 4.3495 | 20.29913 | 44 | 0.33410 | 6212 | 68273 |
| 3 | 3 | 7.9044 | 11.17604 | 20 | 0.33160 | 2744 | 26991 |

# Peak Data List

| peak | 2Theta | d | | FWHM | Intensity | Integrated Int |
|---|---|---|---|---|---|---|
| no. | (deg) | (A) | I/I1 | (deg) | (Counts) | (Counts) |
| 1 | 4.3495 | 20.29913 | 44 | 0.33410 | 6212 | 68273 |
| 2 | 5.8457 | 15.10652 | 100 | 0.40550 | 14036 | 168505 |
| 3 | 7.9044 | 11.17604 | 20 | 0.33160 | 2744 | 26991 |
| 4 | 8.6400 | 10.22611 | 5 | 0.36300 | 709 | 6793 |
| 5 | 8.9975 | 9.82058 | 14 | 0.39580 | 1958 | 16858 |
| 6 | 9.5200 | 9.28272 | 8 | 0.27580 | 1159 | 10258 |
| 7 | 11.6397 | 7.59657 | 18 | 0.39840 | 2557 | 26916 |
| 8 | 12.2000 | 7.24892 | 3 | 0.37220 | 482 | 6182 |
| 9 | 12.6800 | 6.97557 | 5 | 0.35260 | 662 | 6166 |
| 10 | 13.1200 | 6.74261 | 9 | 0.44280 | 1264 | 13808 |
| 11 | 13.7200 | 6.44906 | 5 | 0.40080 | 701 | 8364 |
| 12 | 14.6978 | 6.02215 | 12 | 0.32910 | 1621 | 13247 |
| 13 | 15.1600 | 5.83957 | 4 | 0.35560 | 562 | 5980 |
| 14 | 16.1200 | 5.49390 | 4 | 0.44100 | 564 | 5716 |
| 15 | 16.4000 | 5.40073 | 4 | 0.33740 | 579 | 4433 |
| 16 | 17.6523 | 5.02031 | 6 | 0.94470 | 882 | 21567 |
| 17 | 20.5468 | 4.31915 | 5 | 0.54150 | 714 | 16199 |
| 18 | 23.5200 | 3.77945 | 3 | 0.32680 | 428 | 8157 |
| 19 | 23.8800 | 3.72328 | 6 | 0.32180 | 841 | 10452 |

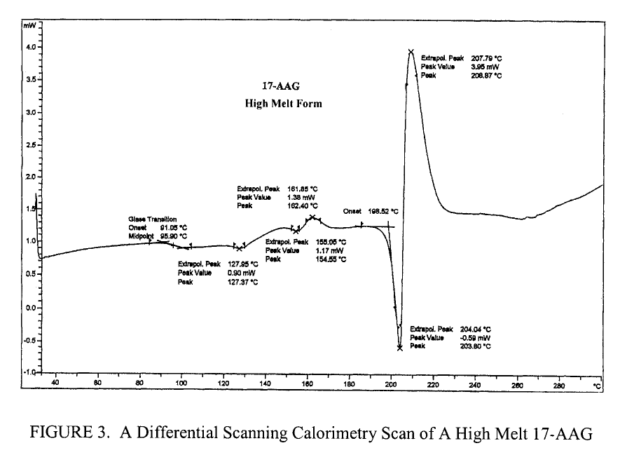

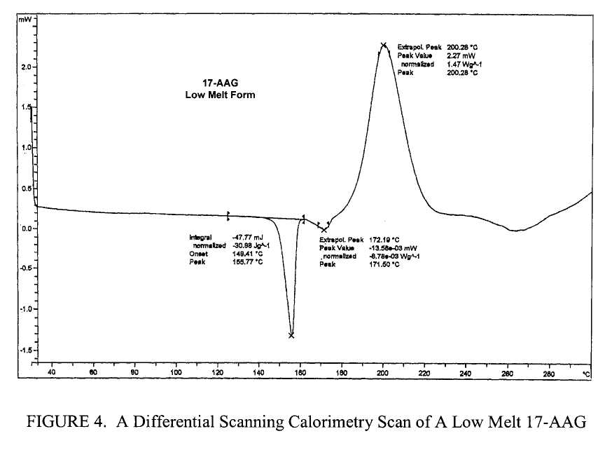

The polymorphic forms of a compound may be characterized by their melting temperatures. Differential scanning calorimetry (DSC) is a common technique used to determine melting temperatures of compounds. FIG. 3 is a DSC scan of the high melt 17-AAG which shows a single endotherm at 204° C. FIG. 4 is a DSC scan of the low melt 17-AAG which shows two distinctive endotherms, a major one centered at 156° C. and a minor one centered at 172° C. Each of the endotherms is indicative of the presence of at least one polymorphic form. Thus, the presence of the two endotherms is indicative that the low melt 17-AAG may be composed of at least two polymorphic forms. Further, the endothermic event terminates at about 176° C. which marks the upper limit of the melting temperature of the low melt polymorphs.

In addition to DSC, other thermal analysis techniques may be used to determine the melting temperatures of the polymorphs; thermal gravimetric analysis (TGA) and capillary melting point are the other common methods used.

The thermal analysis data (i.e., DSC and TGA) of the high melt and low melt forms of 17-AAG summarized in TABLE 3 below. The melting temperatures of the high melt and low melt forms were also analyzed by capillary melting point method and the results are reported in Examples 1-3. It is noted that when comparing the melting temperatures obtained by capillary melting point to those obtained via DSC, there is a discrepancy of a few degrees in each set of the data. This discrepancy can be attributed to the analytical technique used. Capillary melting point measurement depends on visual determination of the onset and completion of the melt cycle, and the very dark colored crystals of 17-AAG make precise determination of these difficult. TABLE 3. Thermal Analysis of High Melt vs Low Melt 17-AAG.

TABLE 3
Thermal Analysis of High Melt vs Low Melt 17-AAG.

| Test | Low Melt Form | High Melt Form |
|---|---|---|
| TGA | No Weight Loss Observed | 3.5% Weight Loss |
| DSC (peak) melt | 156° C. and 172° C. | 204° C. |

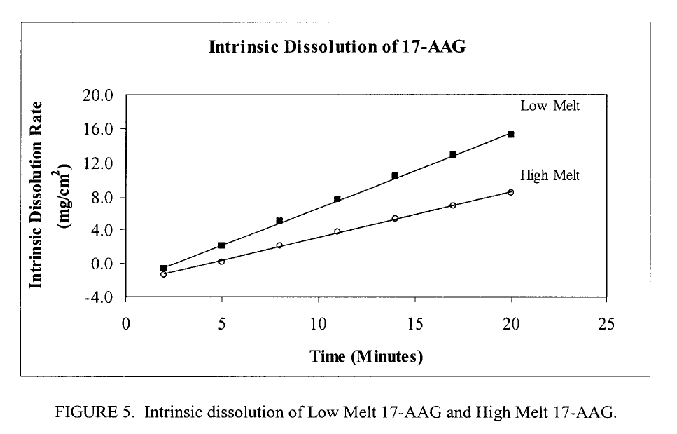

The dissolution rate of an active pharmaceutical ingredient can be affected by its polymorphic state. The intrinsic dissolution rates of the high melt and low melt forms of 17-AAG were determined in ethanol, in which 17-AAG is soluble. The low melt form of 17-AAG had a 60% higher intrinsic dissolution rate (0.885 mg/cm²/min) than the high melt form (0.550 mg/cm²/min), see FIG. 5.

The higher dissolution rate of the low melt form may provide a more efficient manufacturing process. Additionally, the more rapid dissolution may improve the bio-availability of the compound when taken orally, because as the compound is being absorbed from solution in the GI tract, the low melt form has the advantage that it can rapidly dissolve such that a saturated solubility may be maintained and be available for absorption.

The invention contemplates using all the polymorphic forms of the ansamycins, particularly, all the polymorphic forms of 17-AAG, either in a polymorphic mixture or a single polymorph, or amorphous form in the preparation of the formulations.

B. Preparation of the Dosage Formulation

Formulations of the invention may be prepared according to any methods known to the art for the manufacture of pharmaceutical compositions. Generally, the pharmaceutically active compound is dissolved into a crude aqueous phospholipid dispersion followed by reduction of the dispersion particle size. These dispersions can be readily sterilized by filtration, are stable to repeated freeze-thaw cycles, and can also be stored as lyophilizates.

The pH of the formulations of the invention can be manipulated using suitable acids and bases, e.g., hydrochloric acid and sodium hydroxide. Generally, the phospholipid particles are dispersed in a buffered aqueous medium, e.g., sodium acetate buffer. In addition or alternative to the use of sodium acetate, other buffers can be used, e.g., histidine (no more than 5 mM; pH-5), lactic acid (˜10-20 mM; pH˜4), valine (˜10-50 mM; pH-3), etc.

Dispersion and particle size reduction can be effected by a variety of well known techniques, e.g., mechanical mixing, homogenization (e.g., using a polytron or Gaulin high-energy-type instrument), vortexing, and sonication. Sonication can be effected using a bath-type or probe-type instrument. Microfluidizers are commercially available, e.g., from Microfluidics Corp., Newton, Mass., and are further described in U.S. Pat. No. 4,533,254, which make use of pressure-assisted passage across narrow orifices, e.g., as contained in various commercially available polycarbonate membranes. Low pressure devices also exist that can be used. These high and low pressure devices can be used to select for and/or modulate vesicle size. Microchannel filters filter passage under high pressure and can select for a given diameter of disperse particle size. Heat, shaking, and/or sonication can also be used to reduce particle size.

Sterilization of a liquid dispersion can be achieved by various filtration techniques. Filtration can include a pre-filtration through a larger diameter filter, e.g., a 0.45 micron filter, (Gelman mini capsule filter, Pall Corp., East Hills, N.Y., USA) and then through smaller filter, e.g., a 0.2 micron filter. Generally, the filter medium is cellulose acetate (e.g., Sartobran™, Sartorius AG, Goettingen, Germany). A static pressure may be applied to maintain a smooth and continuous flow. Alternatively, the formulation may be directly extruded through a 0.2 micron or smaller filter. In any event, extrusion through a microchannel filter of 0.2 micron or smaller pore size effectively filter-sterilizes, making additional filter-sterilization unnecessary.

Certain embodiments of the formulations and methods of the invention may include lyophilization and rehydration at a suitable point in time. Lyophilization results in a product that is relatively stable and convenient for storage, shipping, and handling. Commercially available rotary evaporation devices exist to accomplish solvent removal. Other devices and methods are known to the skilled artisan. Exemplary conditions for lyophilization can be found in Example 15 but other conditions are known to those of skill in the art. Upon hydration and adjustment to a suitable concentration, administration may be conveniently made to a patient, intravenously or otherwise.

In one embodiment, the active pharmaceutical ingredient, e.g., 17-AAG, is formulated as a 1% (w/w) aqueous phospholipid dispersion. The formulation is prepared by mixing 17-AAG in an aqueous dispersion of phospholipids in a high shear mixture for a short duration and then slowly stirring to remove entrained air. Any phospholipids previously described, such as Phospholipon 90G, phosphatidylcholine, phosphatidylserine, phosphatidylinositol, phosphatidyl ethanolamine, may be used. During the formulation process, other excipients such as buffers, tonicity adjustment agents, and process aids may be added.

The 17-AAG dispersion may be microfluidized to reduce the particle size of the dispersion, typically to less than 200 nm (mean particle size). The dispersions can be filter-sterilized using a sterile 0.2 micron Sartorius Sartobran P capsule filter (500 cm²) (Sartorius AG, Goettingen, Germany), with pressure up to 60 psi used to maintain a smooth and continuous flow. The filtrate can be immediately processed into other formulations such as injectable, oral solutions, tablets or capsules using standard techniques which are known in the art. The filtrate can also be collected, frozen, or lyophilized for future use.

Alternatively, the formulation may be prepared by first preparing a phospholipid dispersion prior to the addition of the pharmaceutically active ingredients as follows. Mix a 1-20% (w/v) phospholipid in sterile water and homogenize the mixture to provide a more uniform dispersion for subsequent microfluidization. The surfactant dispersion may be microfluidized by passage through a Microfluidizer to achieve a particle size of, generally, less than 200 nm and typically between 100-200 nm. The active pharmaceutical ingredients and other excipients are then added and the pH adjusted to between about 5 and 8 using dilute sodium hydroxide and/or hydrocholoric acid, and 10 mM sodium acetate trihydrate, phosphate, or equivalent buffer.

Preparation of specific formulations are discussed in Examples 13 and 14.

III. Characterization and Evaluation of the Drug Formulation

A. Stability Determination of the Active Ingredient Using HPLC

The chemical stability of the active pharmaceutical ingredient, e.g., 17-AAG, can be assessed by high performance liquid chromatographic (HPLC). Specific assay procedures can be developed that allow for the separation of the pharmaceutically active ansamycin from its degradation products. The extent of degradation can be assessed either from the decrease in signal in the HPLC peak associated with the pharmaceutically active ansamycins and/or by the increase in signal in the HPLC peak(s) associated with degradation products. Ansamycins, relative to other components of the formulation, are easily and quite specifically detected at their absorbance maximum of 345 nm.

B. Characterization and Assessment of Chemical and Physical Stability of the Phospholipids

Phospholipids and degradation products may be determined after being extracted from dispersions/emulsions. The lipid mixture can then be separated in a two-dimensional thin-layer chromatographic (TLC) system or in an HPLC system. In TLC, spots corresponding to single constituents can be removed and assayed for phosphorus content. Total phosphorous content in a sample can be quantitatively determined, e.g., by a procedure using a spectrophotometer to measure the intensity of blue color developed at 825 nm against water. In HPLC, phosphatidylcholine (PC) and phosphatidylglycerol (PG) can be separated and quantified with accuracy and precision.

Lipids can be detected in the region of 203-205 nm. Unsaturated fatty acids exhibit high absorbance maxima while the saturated fatty acids exhibit lower absorbance maxima in the 200 nm wavelength region of the UV spectrum. As an example, Vemuri and Rhodes, supra, described the separation of egg yolk PC and PG on Licrosorb Diol and Licrosorb S 1-60. The separations used a mobile phase of acetonitrile-methanol with 1% hexane-water (74:16:10 v/v/v). In 8 minutes, separation of PG from PC was observed. Retention times were approximately 1.1 and 3.2 min, respectively.

C. Evaluation of the Dispersion

Dispersion visual appearance, average droplet size, and size distribution are important parameters to observe and maintain. There are a number of methods to evaluate these parameters. For example, dynamic light scattering and electron microscopy are two techniques that can be used. See, e.g., Szoka and Papahadjopoulos, Annu. Rev. Biophys. Bioeng., 1980 9:467-508. Morphological characterization, in particular, can be accomplished using freeze fracture electron microscopy. Less powerful light microscopes can also be used. The presence of crystalline solid can be determined by polarized light optical microscopy. These microscopic techniques are well known in the art. Dispersion droplet size distribution can be determined, e.g., using a particle size distribution analyzer such as the CAPA-500 made by Horiba Limited (Ann Arbor, Mich., USA), Nanatrac (Mierotrac, Largo, Fla., USA), Coulter Counter (Beckman Coulter Inc., Brea, Calif., USA), or a Zetasizer (Malvern Instruments, Southborough, Mass., USA).

IV. Other Modes of Formulation and Administration

A. Other Formulations

Although intravenous administration is described in various aspects and embodiments of the invention, one of ordinary skill will appreciate that the methods can be modified or readily adapted to accommodate other administration modes, e.g., oral, aerosol, parenteral, subcutaneous, intramuscular, intraperitoneal, rectal, vaginal, intratumoral, or peritumoral. The following discussion is largely known to the person of skill but is nevertheless provided as a backdrop to illustrate other possibilities for the invention. It will be appreciated that following the discussion duplicates in part previous discussions included herein.

Pharmaceutical compositions may be manufactured utilizing a conventional mixing, dissolving, granulating, dragee-making, levigating, emulsifying, encapsulating, entrapping or lyophilizing processes.

Pharmaceutically acceptable compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Some excipients and their use in the preparation of formulations have already been described. Others are known in the art, e.g., as described in PCT/US99/30631, REMINGTON'S PHARMACEUTICAL SCIENCES, Meade Publishing Co., Easton, Pa. (most recent edition), and Goodman and Gilman's THE PHARMACEUTICAL BASIS OF THERAPEUTICS, Pergamon Press, New York, N.Y. (most recent edition).

For injection, the agents may be formulated in aqueous solutions, generally in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Formulations of the invention, as described previously, and upon hydration of the lyophilized cakes, are well suited for immediate or near-immediate parenteral administration by injection, e.g., by bolus injection or continuous infusion. Formulations for injection may be presented in unit dosage form, e.g., in ampoules or in multi-dose containers with an added preservative. As discussed previously, lyophilized products are embodiments for the invention; and ampoules or other packaging, optionally light-resistant, may contain this lyophilized product, which may then be conveniently (re)hydrated prior to administration to a patient.

B. Dose Range

A phase I pharmacologic study of 17-AAG in adult patients with advanced solid tumors determined a maximum tolerated dose (MTD) of 40 mg/m² when administered daily by 1-hour infusion for 5 days every three weeks. Wilson et al., 2001 Am. Soc. Clin. Oncol., abstract, Phase I Pharmacologic Study of 17-(Allylamino)-17-Demethoxygeldanamycin (AAG) in Adult Patients with Advanced Solid Tumors. In this study, mean +/−SD values for terminal half-life, clearance and steady-state volume were determined to be 2.5±0.5 hours, 41.0±13.5 L/hour, and 86.6+34.6 L/m², respectively. Plasma Cmax levels were determined to be 1860+/−660 nM and 3170+/−1310 nM at 40 and 56 mg/m². Using these values as guidance, it is anticipated that the range of useful patient dosages for formulations of the present invention will include between about 0.40 mg/m² and 400 mg/m² of active ingredient, wherein m² represents surface area. Standard algorithms exist to convert mg/m² to mg drug/kg bodyweight.

### EXAMPLES

### Example 1

### Preparation of 17-AAG

To 45.0 g (80.4 mmol) of geldanamycin in 1.45 L of dry THF in a dry 2 L flask was added drop-wise over 30 minutes 36.0 mL (470 mmol) of allyl amine in 50 mL of dry THF. The reaction mixture was stirred at room temperature under nitrogen for 4 hr at which time TLC analysis indicated the reaction was complete [(GDM: bright yellow: Rf=0.40; (5% MeOH-95% CHCl₃); 17-AAG: purple: Rf=0.42 (5% MeOH-95% CHCl₃)]. The solvent was removed by rotary evaporation and the crude material was slurried in 420 mL of H₂O:EtOH (90:10) at 25° C., filtered and dried at 45° C. for 8 hr to give 40.9 g (66.4 mmol) of 17-AAG as purple crystals (82.6% yield, >98% pure by HPLC monitored at 254 nm). m.p. 206-212° C. ¹H NMR and HPLC are consistent with the desired product.

### Example 2

### Preparation of a Low Melt Form of 17-AAG

The crude 17-AAG from Example 1 was dissolved in 800 mL 2-propyl alcohol (isopropanol) at 80° C. and then cooled to room temperature. Filtration followed by drying at 45° C. for 8 hr gave 44.6 g (72.36 mmol) of 17-AAG as purple crystals (90% yield, >99% pure by HPLC monitored at 254 nm). m.p.=147-153° C. ¹H NMR and HPLC are consistent with the desired product.

### Example 3

### Solvant Stability of a Low Melt Form of 17-AAG

The 17-AAG product from Example 2 was dissolved in 400 mL of H₂O:EtOH (90:10) at 25° C. Filltration followed by aging at 45° C. for 8 hr gave 42.4 g (68.6 mmol) of 17-AAG as purple crystals (95% yield, >99% pure by HPLC monitored at 254 nm). m.p.=147-175° C. ¹H NMR and HPLC are consistent with the desired product.

### Example 4

### Preparation of Compound 237: A Dimer

3,3-diamino-dipropylamine (1.32 g, 9.1 mmol) was added dropwise to a solution of geldanamycin (10 g, 17.83 mmol) in DMSO (200 mL) in a flame-dried flask under N₂ and stirred at room temperature. The reaction mixture was diluted with water after 12 hours. A precipitate was formed and filtered to give the crude product. The crude product was chromatographed by silica chromatography (5% CH₃OH/CH₂Cl₂) to afford the desired dimer as a purple solid. The pure purple product was obtained after flash chromatography (silica gel); yield: 93%; m.p. 165° C.; ¹H NMR (CDCl₃) 0.97 (d, J=6.6 Hz, 6H, 2CH₃), 1.0 (d, J=6.6 Hz, 6H, 2CH₃), 1.72 (m, 4H, 2 CH₂), 1.78 (m, 4H, 2CH₂), 1.80 (s, 6H, 2CH₃), 1.85 (m, 2H, 2CH), 2.0 (s, 6H, 2CH₃), 2.4 (dd, J=11 Hz, 2H, 2CH), 2.67 (d, J=15 Hz, 2H, 2CH), 2.63 (t, J=10 HZ, 2H, 2CH), 2.78 (t, J=6.5 Hz, 4H, 2CH₂), 3.26 (s, 6H, 20CH₃), 3.38 (s, 6H, 20CH₃), 3.40 (m, 2H, 2CH), 3.60 (m, 4H, 2CH₂), 3.75 (m, 2H, 2CH), 4.60 (d, J=10 Hz, 2H, 2CH), 4.65 (Bs, 2H, 20H), 4.80 (bs, 4H, 2NH₂), 5.19 (s, 2H, 2CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.17 (m, 2H, 2NH), 7.24 (s, 2H, 2CH═), 9.20 (s, 2H, 2N—H); MS (m/z) 1189 (M+H).

The corresponding HCl salt was prepared by the following method: an HCl solution in EtOH (5 ml, 0.12 3N) was added to a solution of Compound 237 (1 g as prepared above) in THF (15 ml) and EtOH (50 ml) at room temperature. The reaction mixture was stirred for 10 min. The salt was precipitated, filtered and washed with a large amount of EtOH and dried in vacuo. Alternatively, a “mesylate” salt can be formed using methanesulfonic acid instead of HC1.

### Example 5

### Preparation of Compound 914

To geldanamycin (500 mg, 0.89 mmol) in 10 mL of dioxane was added selenium (IV) dioxide (198 mg, 1.78 mmol) at room temperature. The reaction mixture was heated to 100° C. and stirred for 3 hours. After cooling to room temperature, the solution was diluted with ethyl acetate, washed with water and brine, dried over magnesium sulfate, filtered and evaporated in vacuo. The final pure yellow product was obtained after column chromatography (silica gel); yield: 75%; ¹H NMR (CDCl₃) δ 0.97(d, J=7.0 Hz, 3H, CH₃), 1.01(d, J=7.0 Hz, 3H, CH₃), 1.75(m, 3H, CH₂+CH), 2.04(s, 3H, CH₃), 2.41(d, J=9.9 Hz, 1H, CH₂), 2.53(t, J=9.9 Hz, 1H, CH₂), 2.95(m, 1H, CH), 3.30(m, 2H, CH+OH), 3.34(s, 6H, 2CH₃), 3.55(m, 1H, CH), 4.09(m, 1H, CH₂), 4.15(s, 3H, CH₃), 4.25(m, 1H, CH₂), 4.41(d, J=9.8 Hz, 1H, CH), 4.80(bs, 2H, CONH₂), 5.32(s, 1H, CH), 5.88(t, J=10.4 Hz, 1H, CH═), 6.04(d, J=9.7 Hz, 1H, CH═), 6.65(t, J=11.5 Hz, 1H, CH═), 6.95(d, J1=1.5 Hz, 1H, CH═), 7.32(s, 1H, CH—Ar), 8.69(s, 1H, NH); MS (m/z) 575.6 (M−1).

### Example 6

### Preparation of Compound 967

To Compound 914 (50 mg, 0.05 mmol) in 3 mL of THF was added allylamine (3.5 mg, 0.06 mmol). The reaction mixture was stirred at room temperature for 24 hours. The solvent was removed by rotary evaporation. The pure purple product was obtained after column chromatography (silica gel); yield: 90%; ¹H NMR (CDCl₃) δ 0.89(d, J=6.6 Hz, 3H, CH₃), 1.03 (d, J=6.9 Hz, 3H, CH₃), 1.78(m, 1H, CH), 1.82(m, 2H, CH₂), 2.04 (s, 3H, CH₃), 2.37(dd, J=13.7 Hz, 1H, CH₂), 2.65(d, J=13.7 Hz, 1H, CH₂), 2.90(m, 1H, CH), 3.33(s, 3H, CH₃), 3.34(s, 3H, CH₃), 3.45(m, 2H, CH+OH), 3.58(m, 1H, CH), 4.14(m, 3H, CH₂+CH₂), 4.16(m, 1H, CH₂), 4.42(s, 1H, OH), 4.43(d, J=1OHz, 1H, CH), 4.75(bs, 2H, CONH₂), 5.33(m, 2H, CH₂═), 5.35(s, 1H, CH), 5.91(m, 2H, CH=+CH═), 6.09(d, J=9.9 Hz, 1H, CH═), 6.46(t, J=5.8 Hz, 1H, NH), 6.66(t, J=11.6 Hz, 1H, CH═), 6.97(d, J=11.6 Hz, 1H, CH═), 7.30(s, 1H, CH), 9.15(s, 1H, NH).

### Example 7

### Preparation of Compound 956

Compound 956 was prepared by the same method described for Compound 967 except that azetidine was used instead of allylamine. The final pure purple product was obtained after column chromatography (silica gel); yield: 89%; ¹H NMR (CDCl₃) δ 0.99 (d, J=6.8 Hz, 3H, CH₃), 1.04 (d, J=6.8 Hz, 3H, CH₃), 1.77 (m, 1H, CH), 1.80 (m, 2H, CH₂), 2.06 (s, 3H, CH₃), 2.26 (m, 1H, CH₂), 2.50(m, 2H, CH₂), 2.67 (d, 1H, CH₂), 2.90 (m, 1H, CH), 3.34 (s, 3H, CH₃), 3.36 (s, 3H, CH₃), 3.48 (m, 2H, OH+CH), 3.60 (t, J=6.8 Hz, 1H, CH), 4.11 (dd, J=12 Hz, J=4.5 Hz, 1H, CH₂), 4.30 (dd, J=12 Hz, J=4.5 Hz, 1H, CH₂), 4.45 (d, J=10.0 Hz, 1H, CH), 4.72 (m, 5H, 2CH₂+OH), 4.78 (bs, 2H, NH₂), 5.37 (s, 1H, CH), 5.89 (t, J=10.5 Hz, 1H, CH═), 6.10 (d, J=10 Hz, 1H, CH═), 6.66 (t, J=12 Hz, 1 H, CH═), 7.00 (d, J=12 Hz, 1H, CH═), 7.17 (s, 1H, CH═), 9.20 (s, 1H, CONH); MS(m/z) 602 (M+1).

### Example 8

### Preparation of Compound 529

A solution of 17-aminogeldanamycin (1 mmol) in EtOAc was treated with Na₂S₂O₄ (0.1 M, 300 ml) at room temperature. After 2 h, the aqueous layer was extracted twice with EtOAc and the combined organic layers were dried over Na₂SO₄, concentrated under reduce pressure to give 18,21-dihydro-17-aminogeldanamycin as a yellow solid. This solid was dissolved in anhydrous THF and transferred via cannula to a mixture of picolinoyl chloride (1.1 mmol) and MS4A (1.2 g). Two hours later, EtN(i-Pr)₂ (2.5 mmol) was further added to the reaction mixture. After overnight stirring, the reaction mixture was filtered and concentrated under reduce pressure. Water was then added to the residue, which was extracted with EtOAc three times; the combined organic layers were dried over Na₂SO₄ and concentrated under reduce pressure to give the crude product which was purified by flash chromatography to give 17-picolinoyl-aminogeldanamycin, Compound 529, as a yellow solid. Rf=0.52 in 80:15:5 CH₂Cl₂: EtOAc: MeOH. m.p.=195-197° C. ¹H NMR (CDCl₃) δ 0.91 (d, 3H), 0.96 (d, 3H), 1.71-1.73 (m, 2H), 1.75-1.79 (m, 4H), 2.04 (s, 3H), 2.70-2.72 (m, 2H), 2.74-2.80 (m, 1H), 3.33-3.35 (m, 7H), 3.46-3.49 (m, 1H), 4.33 (d, 1H), 5.18 (s, 1H), 5.77 (d, 1H), 5.91 (t, 1H), 6.57 (t, 1H), 6.94 (d, 1H), 7.51-7.56 (m, 2H), 7.91 (dt, 1H), 8.23 (d, 1H), 8.69-8.70 (m, 1H), 8.75(s, 1H), 10.51 (s, 1H).

### Example 9

### Preparation of Compound 1046

Compound 1046 was prepared according to the procedure described for Compound 529 using 4-chloromethyl-benzoyl chloride instead of picolinoyl chloride. (3.1 g, 81%). Rf=0.45 in 80:15:5 CH₂Cl₂: EtOAc: MeOH. ¹H NMR CDCl₃ δ 0.89 (d, 3H), 0.93 (d, 3H), 1.70 (br s, 2H), 1.79 (br s, 4H), 2.04 (s, 3H), 2.52-2.58 (m, 2H), 2.62-2.63 (m, 1H), 2.76-2.79 (m, 1H), 3.33 (br s, 7H), 3.43-3.45 (m, 1H), 4.33 (d, 1H), 4.64 (s, 2H), 5.17 (s, 1H), 5.76 (d, 1H), 5.92 (t, 1H), 6.57 (t, 1H), 6.94 (d, 1H), 7.49 (s, 1H), 7.55 (d, 2H), 7.91 (d, 2H), 8.46 (s, 1H), 8.77 (s, 1H).

### Example 10

### Preparation of Compound 1059

To a solution of Compound 1046 (0.14 g, 0.2 mmol) in THF (5 ml) were added sodium iodide (30 mg, 0.2 mmol) and morpholine (35 μL, 0.4 mmol). The resulting mixture was heated at reflux for 10 h whereupon it was cooled to room temperature and concentrated under reduce pressure. The residue was redissolved in EtOAc (30 ml), washed with water (10 ml), dried with Na₂SO₄ and concentrated again. The residue was then recrystallized in EtOH (10 ml) to give Compound 1059 as a yellow solid (100 mg, 66%). Rf=0.10 in 80:15:5 CH₂Cl₂: EtOAc: MeOH. ¹H NMR CDCl₃ δ 0.93 (s, 3H), 0.95 (d, 3H), 1.70 (br s, 2H), 1.78 (br s, 4H), 2.03 (s, 3H), 2.48 (br s, 4H), 2.55-2.62 (m, 3H), 2.74-2.79 (m, 1H), 3.32 (br s, 7H), 3.45 (m, 1H), 3.59 (s, 2H), 3.72-3.74 (m, 4H), 4.32 (d, 1H), 5.15 (s, 1H), 5.76 (d, 1H), 5.91 (t, 1H), 6.56 (t, 1H), 6.94 (d, 1H), 7.48 (s, 1H), 7.50 (d, 2H), 7.87 (d, 2H), 8.47 (s, 1H), 8.77 (s, 1H).

### Example 11

### Preparation of Compound 1236

Compound 1236 was prepared according to the procedure described for Compound 1059 using benzylethyl amine instead of morpholine. Rf=0.43 in 80:15:5 CH₂Cl₂: EtOAc: MeOH. ¹H NMR CDCl₃ δ 0.925 (s, 3H), 0.95 (d, 3H), 1.09 (t, 3H), 1.70 (br s, 2H), 1.79 (br s, 4H), 2.04 (s, 3H), 2.50-2.62 (m, 5H), 2.75-2.79 (m, 1H), 3.32 (br s, 7H), 3.46 (m, 1H), 3.59 (s, 2H), 3.63 (s, 2H), 4.33 (d, 1H), 5.16 (s, 1H), 5.78 (d, 1H), 5.91 (t, 1H), 6.57 (t, 1H), 6.94 (d, 1H), 7.25-7.27 (m, 1H), 7.32-7.38 (m, 4H), 7.48 (s, 1H), 7.53 (d, 2H), 7.85 (d, 2H), 8.47 (s, 1H), 8.77 (s, 1H).

### Example 12

### Preparation of Compound 563: 17-(benzoyl)-aminogeldanamycin

A solution of 17-aminogeldanamycin (1 mmol) in EtOAc was treated with Na₂S₂O₄ (0.1 M, 300 mL) at room temperature. After 2 h, the aqueous layer was extracted twice with EtOAc and the combined organic layers were dried over Na₂SO₄, concentrated under reduce pressure to give 18,21-dihydro-17aminogeldanamycin as a yellow solid. This solid was dissolved in anhydrous THF and transferred via cannula to a mixture of benzoyl chloride (1.1 mmol) and MS4A (1.2 g). Two hours later, EtN(i-Pr)₂ (2.5 mmol) was further added to the reaction mixture. After overnight stirring, the reaction mixture was filtered and concentrated under reduce pressure. Water was then added to the residue which was extracted with EtOAc three times, the combined organic layers were dried over Na₂SO₄ and concentrated under reduce pressure to give the crude product which was purified by flash chromatography to give 17-(benzoyl)-aminogeldanamycin. Rf=0.50 in 80:15:5 CH₂Cl₂: EtOAc: MeOH. m.p.=218-220° C. ¹H NMR (CDCl₃) 0.94 (t, 6H), 1.70 (br s, 2H), 1.79 (br s, 4H), 2.03 (s, 3H), 2.56 (dd, 1H), 2.64 (dd, 1H), 2.76-2.79 (m, 1H), 3.33 (br s, 7H), 3.44-3.46 (m, 1H), 4.325 (d, 1H), 5.16 (s, 1H), 5.77 (d, 1H), 5.91 (t, 1H), 6.57 (t, 1H), 6.94 (d, 1H), 7.48 (s, 1H), 7.52 (t, 2H), 7.62 (t, 1H), 7.91 (d, 2H), 8.47 (s, 1H), 8.77 (s, 1H).

### Example 13

### Specific Formulation Embodiments

1-20% (w/v) phospholipids-surfactant/aqueous solutions were prepared in sterile water for injection. The phospholipids/aqueous solutions were homogenized to provide a more uniform dispersion for subsequent microfluidization. The surfactant dispersion was then microfluidized by passage through a Model 11OS microfluidizer (Microfluidics Inc., Newton, Mass., USA) operated at a static pressure of about 110 psi (operating pressure of 60-95 psi). Drugs were dissolved in the phospholipid/aqueous solution (1-20 mg/mL) at mole ratios ranging from 1:1 to 1:20 (drug:phopholipid solution). The drugs used were Compound 237, Compound 956 and Compound 1236 and pharmaceutically acceptable salts and prodrugs, thereof. Sucrose, mannitol and/or dextrose were added in the range of 110% w/v and the pH adjusted to between about 5 and 8 using dilute sodium hydroxide and/or hydrocholoric acid, and 10 mM sodium acetate trihydrate, phosphate, or equivalent buffer. The mean particle size of the drug:phospholipid complexes is between about 20-150 nm as determined by laser-light scattering techniques. The dispersions was passed across a 0.45 micron Gelman mini capsule filter (Pall Corp., East Hills, N.Y., USA), and then across a sterile 0.2 micron Sartorius Sartobran P capsule filter (500 cm2) (Sartorius AG, Goettingen, Germany). Pressure up to 60 PSi was used to maintain a smooth and continuous flow. Specific formulations made within these embodiment parameters included: A 6.2% phospholipid-surfactant (Phospholipon 90) dispersion solution having ˜2-8 mg/mL drug (drug:phospholipid molar ratios of 1:8 to 1:20), 10% sucrose, and buffered to pH 5 or 7 using 10 mM sodium acetate trihydrate buffer and/or dilute sodium hydroxide. A 1.2% phospholipid-surfactant (Phospholipon 90) dispersion solution having 2 mg/mL drug (molar ratios of 1:10 drug:phopholipids) and 1% mannitol, 5% dextrose, and buffered to pH 5 using 10 mM sodium acetate trihydrate buffer and dilute hydrochloric acid. A 2-4 mg/mL solution of drug dissolved in TWEEN® 80 surfactant at drug: TWEEN® 80 molar ratios of 1:5 to 1:20, adjusted to 7.0 or buffered to pH 7.2 using 10 mM phosphate buffer.

A 13.2% w/v solution of Poloxamer 188 was prepared having at a drug:Poloxomer molar ratio of 1:5 (final concentration of drug 4 mg/mL), and buffered to pH 7 using 10 mM phosphate buffer.

### Example 14

### Preparation of a 17-AAG Aqueous Phospholipid Dispersion

17-AAG was formulated as a 1% (w/w) aqueous phospholipid dispersion. L-histidine and sucrose were dissolved in water. The phospholipids were added and a high-shear mixer was used to disperse the phospholipids for five minutes at about 3500 rpm. 17-AAG was added to the phospholipid dispersion and mixed with the high-shear mixer to mix/disperse 17-AAG in the phospholipids. The product are removed from the high shear mixer, then slowly mixed (no vortex) to allow most of the entrained air to escape. Then 0.7 g of a 50/50 (w/w) mixture of ethanol and TWEEN® 80 were added to the stirring 17-AAG dispersion and mixed for one hour to allow more entrained air to escape. The 17-AAG dispersion was microfluidized at 16-19,000 psi to reduce the particle size of the dispersion from about 5 μm to 0.1-0.5 μm (mean particle size). The formulation composition was below:

| Ingredient | % by weight | Function |
|---|---|---|
| 17-AAG | 1.0 | Active ingredient |
| L-histidine | 0.1 | Buffer |
| Sucrose | 7.5 | Tonicity/cryoprotectant |
| Phospholipid | 18.8 | Lipid complex/carrier |
| Ethanol | 0.5 | Process aid |
| Tween 80 | 0.5 | Process aid |
| Water | 71.5 | Diluent |

### Example 15

### Lyophilization

Illustrative lyophilization schemes that can be used include that described in the following Table.

| Initial | Final | Pressure | |
|---|---|---|---|
| Temp. (° C.) | Temp. (° C.) | (mTorr) | Action |
| 25 | −40 | Ambient | Ramp at 1° C./min |
| −40 | −40 | Ambient | Hold for 60 min |
| −40 | −40 | 50 | Condenser at−60° C. to −80° C. |
| −40 | −28 | | Ramp at 1° C./min |
| −28 | −28 | 50 | Hold for 7200 min |
| −28 | 30 | 50 | Ramp at 1° C./min |
| 30 | 30 | 50 | Hold for 300 min |

Complete
Stopper vials under N₂ at approximately 0.9 atm

### Example 16

### Intravenous Injection and Tolerance

A water soluble salt of a poorly water soluble ansamycin (e.g., Compound 237-mesylate) when formulated in an aqueous solution was found to be irritating to rat tail vein upon intravenous infusion. The dispersion formulation of Compound 237-mesylate described above produced no evidence of vein irritation when given at the same dose and over the same infusion interval as the solution formulation. Pharmacokinetics for the solution formulation and the dispersion formulation were very similar. The method of the invention was also used to prepare dispersion formulations with the hydrochloride and phosphate salts of Compound 237. These formulations were also much better tolerated than the aqueous solution of Compound 237. Dispersion formulations were also prepared using water soluble and slightly water soluble derivatives of geldanamycin. Specifically, similar dispersions containing DMAG were similarly formulated and well-tolerated upon tail vein injection into mice and rats.

The foregoing examples are not intended to be limiting of and are merely representative of various embodiments of the invention. It will be readily apparent to one skilled in the art that varying substitutions and modifications may be made to the invention without departing from the scope and spirit of the invention. Thus, such additional embodiments are within the scope of the invention and the following claims. The reagents described herein are either commercially available, e.g., from Sigma-Aldrich, or else readily producible without undue experimentation using routine procedures known to those of ordinary skill in the art and/or described in publications herein incorporated by reference.

What is claimed:

1. A pharmaceutical formulation comprising aqueous dispersible particles, comprising
an ansamycin, or a polymorph, solvate, ester, tautomer, enantiomer, pharmaceutically acceptable salt or prodrug thereof; and
a pharmaceutically acceptable phospholipid;
wherein said formulation is substantially devoid of medium and long chain triglycerides, and
said phospholipid is present at a concentration of at least 5% w/w of said formulation.

2. The pharmaceutical formulation of claim 1 wherein said medium and long chain triglycerides are present at a combined concentration of about 1% w/v or less.

3. The pharmaceutical formulation of claim 1 wherein said ansamycin is selected from the group consisting of:

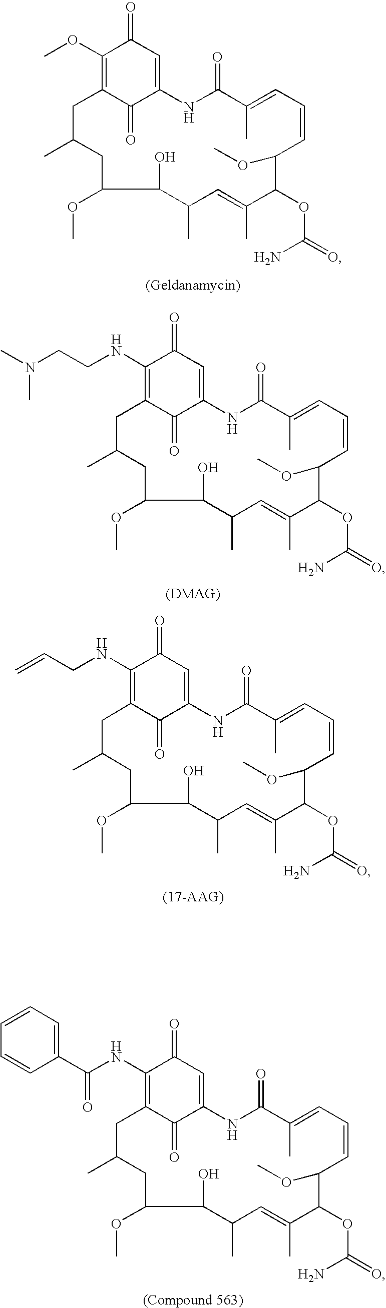

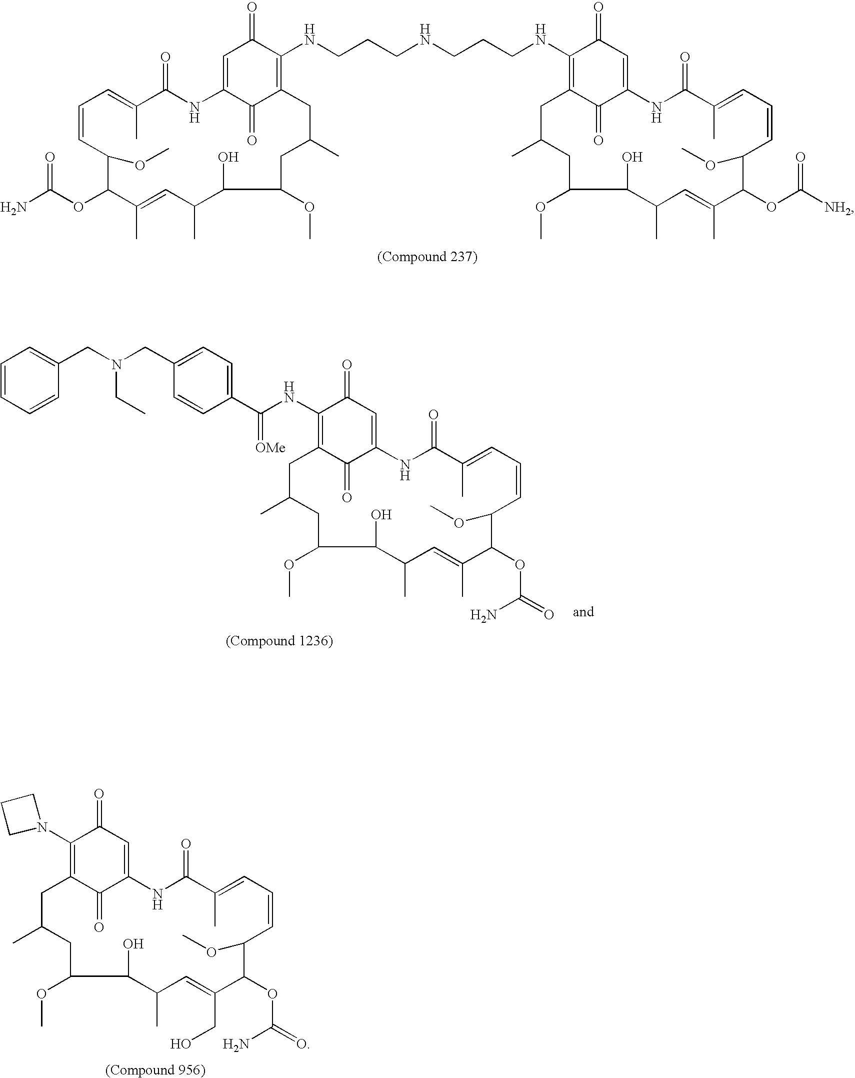

4. The pharmaceutical formulation of claim 1 wherein said ansamycin is 17-AAG.

5. The pharmaceutical formulation of claim 4 wherein said 17-AAG is a high melt 17-AAG, a low melt 17-AAG, an amorphous form of 17-AAG or any combination thereof.

6. The pharmaceutical formulation of claim 1 wherein said ansamycin comprises low melt forms of 17-AAG characterized by DSC melting temperatures below 175° C. and by an X-ray powder diffraction pattern comprising peaks located at 5.85 degree, 4.35 degree and 7.90 degree two-theta angles.

7. The pharmaceutical formulation of claim 1 wherein said ansamycin comprises a low melt polymorph of 17-AAG characterized by a DSC melting temperature of about 156° C. and by an X-ray powder diffraction pattern comprising peaks located at 5.85 degree, 4.35 degree and 7.90 degree two-theta angles.

8. The pharmaceutical formulation of claim 1 wherein said ansamycin is a low melt polymorph of 17-AAG characterized by a DSC melting temperature of about 172° C.

9. The pharmaceutical formulation of claim 1 wherein said ansamycin is a pharmaceutically acceptable hydrochloride or phosphate salt of 17-AAG.

10. The pharmaceutical formulation of claim 1 wherein the concentration of said ansamycin, or polymorph, solvate, ester, tautomer, enantiomer, pharmaceutically acceptable salt or prodrug thereof, in said formulation is at least 0.5 mg/mL.

11. The pharmaceutical formulation of claim 1 wherein the concentration of said ansamycin, or polymorph, solvate, ester, tautomer, enantiomer, pharmaceutically acceptable salt or prodrug thereof, in said formulation is at least 5.0 mg/mL.

12. The pharmaceutical formulation of claim 1 wherein the concentration of said ansamycin, or polymorph, solvate, ester, tautomer, enantiomer, pharmaceutically acceptable salt or prodrug thereof, in said formulation is at least 50 mg/mL.

13. The pharmaceutical formulation of claim 1 wherein said dispersible particles have been treated to reduce particle size, wherein said treatment comprises sonication, high shear homogenization, microfluidization, extrusion through controlled pore size filters, or any combination thereof.

14. The pharmaceutical formulation of claim 1 wherein the particle size of said aqueous dispersible particles is from about 100 nm to about 200 nm.

15. The pharmaceutical formulation of claim 1 wherein the particle size of said aqueous dispersible particles is about 200 nm or less.

16. The pharmaceutical formulation of claim 1 wherein said aqueous dispersible particles are colloidal.

17. The pharmaceutical formulation of claim 1 wherein said phospholipid comprises phosphatidylcholine, phosphatidalserine, phosphatidylinositol, phosphatidalethanolamine, Phospholipon 90G or any combination thereof.

18. The pharmaceutical formulation of claim 1, further comprising one or more excipients.

19. The pharmaceutical formulation of claim 18, wherein said one or more excipients comprise a cryoprotectant, a tonicity modifier, a bulking agent or any combination thereof.

20. A method of treating or preventing a disorder in a mammal, comprising administering to said mammal a pharmaceutically effective amount of a pharmaceutical formulation of claim 1.

21. The method of claim 20 wherein said disorder involves ischemia, a proliferative disorder, infection, acquired immunodeficiency syndrome, a neurological disorder, a tumor, leukemia, chronic lymphocytic leukemia, a neoplasm, cancer, a carcinoma or other malignant diseases.

22. The method of claim 21 wherein said proliferative disorder is selected from the group consisting of tumors, inflammatory diseases, fungal infection, yeast infection, and viral infection.

23. The method of claim 20 wherein the concentration of said ansamycin, or polymorph, solvate, ester, tautomer, enantiomer, pharmaceutically acceptable salt or prodrug thereof, in said formulation is from about 1% to about 1.5% (w/w).

24. The method of claim 20 wherein the concentration of said ansamycin, or polymorph, solvate, ester, tautomer, enantiomer, pharmaceutically acceptable salt or prodrug thereof, in said formulation is from about 0.5 mg/ml to about 50 mg/ml.

25. The method of claim 20 wherein said ansamycin is selected from the group consisting of:

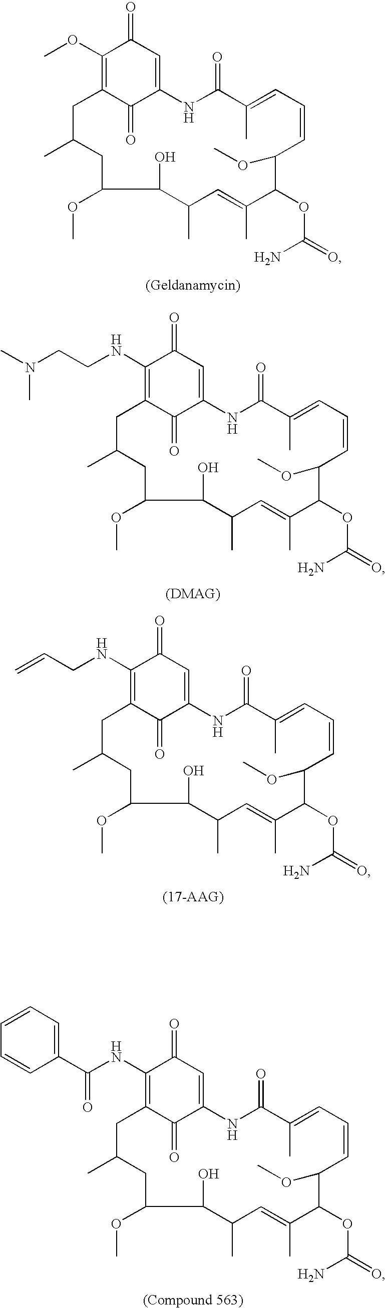

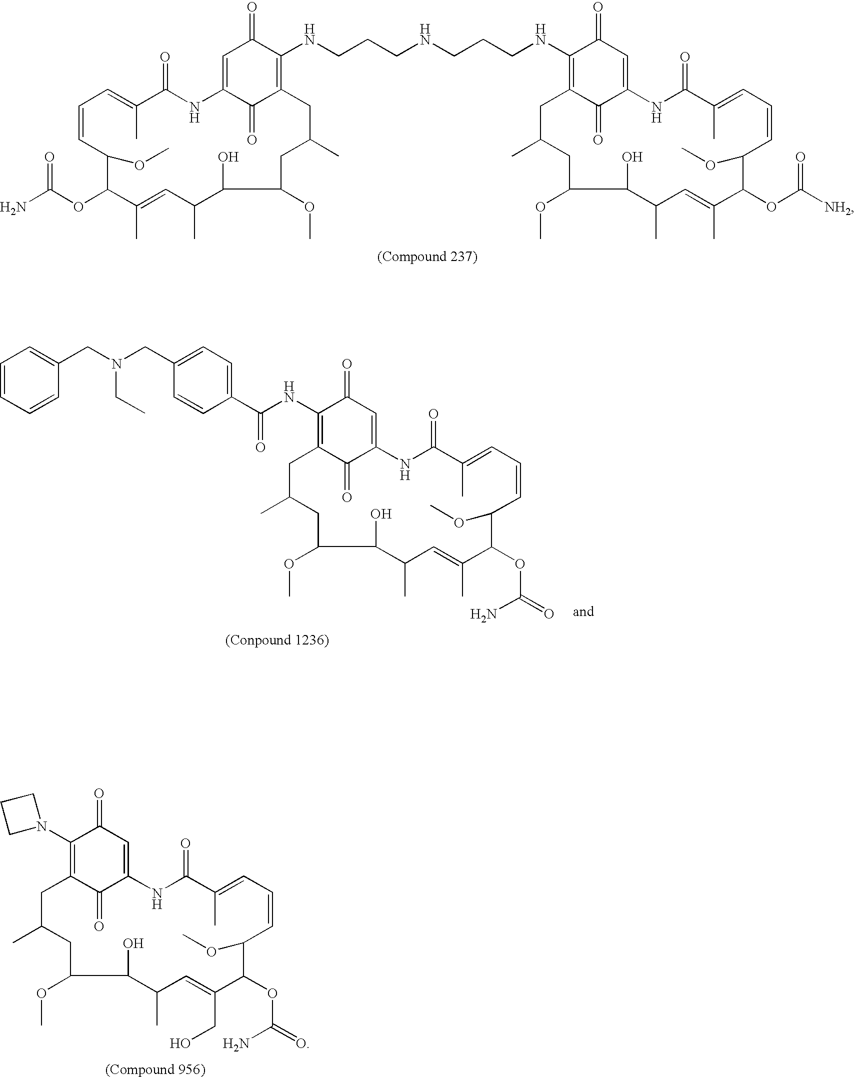

26. The method of claim 20 wherein said ansamycin is 17-AAG.

27. The method of claim 26 wherein said 17-AAG is a high melt 17-AAG, a low melt 17-AAG, an amorphous form of 17-AAG or any combination thereof.

28. The method of claim 26 wherein said 17-AAG comprises a low melt form of 17-AAG.

29. The use of the pharmaceutical formulation of claim 1 in the manufacture of a medicament.

30. The use of claim 29 wherein said medicament is for the therapeutic or prophylactic treatment of HSP90 mediated diseases.

31. A method of preparing a pharmaceutical formulation, comprising:
(a) forming dispersion particles comprising
an ansamycin, or a polymorph, solvate, ester, tautomer, enantiomer, pharmaceutically acceptable salt or prodrug thereof; and
a pharmaceutically acceptable phospholipid;
(b) optionally reducing the size of said dispersion particles;
(c) optionally freezing the product of step (a) or (b);
(d) optionally thawing the product of step (c);
(e) optionally lyophilizing the product of any of steps (a)-(d); and
(f) optionally rehydrating the product of step (e); and
wherein said formulation is substantially devoid of medium and long chain triglycerides.

32. The method of claim 31 wherein said medium and long chain triglycerides are present at a combined concentration of about 1% w/v or less.

33. The method of claim 31 wherein said ansamycin is selected from the group consisting of:

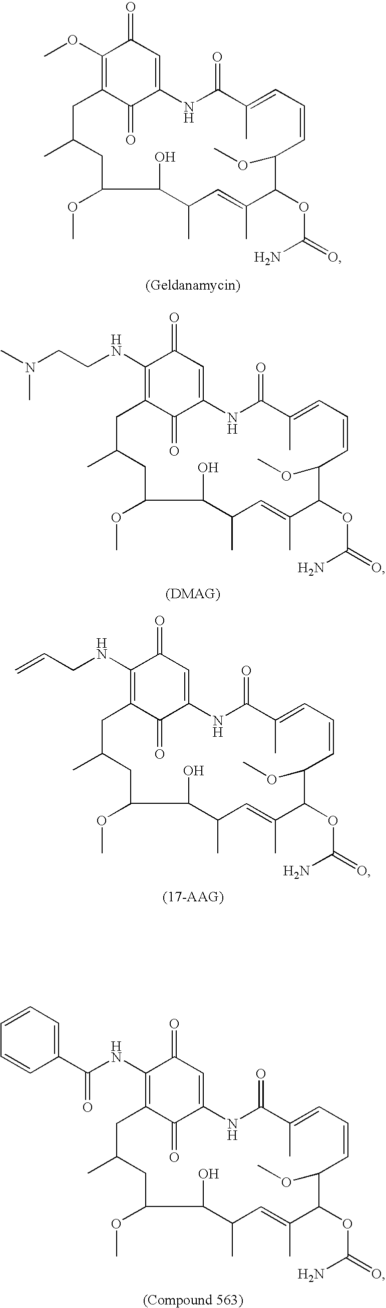

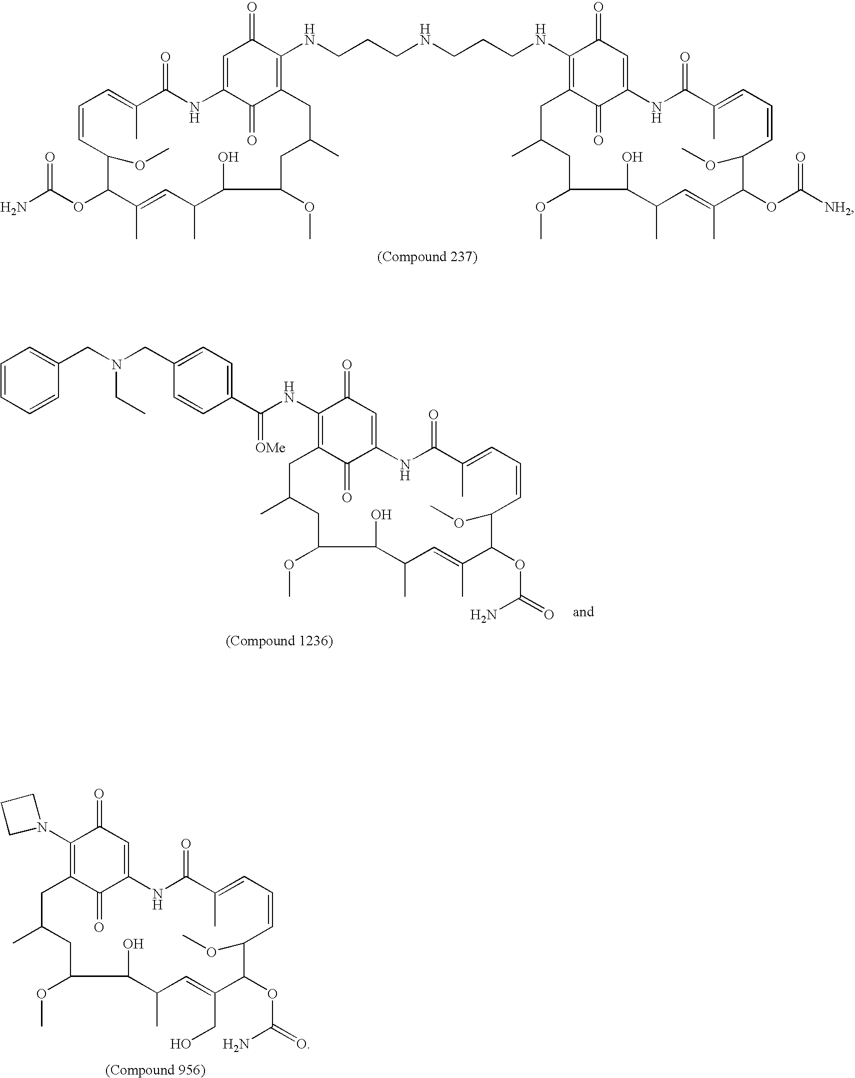

34. The method of claim 31 wherein said ansamycin is 17-AAG.

35. The method of claim 34 wherein said 17-AAG is a high melt 17-AAG, a low melt 17-AAG, an amorphous form of 17-AAG or any combination thereof.

36. The method of claim 31 wherein said ansamycin comprises a low melt form of 17-AAG.

37. The method of claim 31 wherein said phospholipid comprises phosphatidylcholine, phosphatidylserine, phosphatidylinositol, phosphatidylethanolamine, Phospholipon 90G or any combination thereof.

38. The method of claim 31 wherein said phospholipid comprises Phospholipon 90G.

39. The method of claim 31 wherein said reducing step is present and comprises sonication, high shear homogenization, microfluidization, extrusion through controlled pore size filters or any combination thereof.

40. The method of claim 31 wherein the particle size of said dispersion particles is about 200 nm or less.

41. The method of claim 31 wherein said formulation is a colloidal dispersion.

42. The method of claim 31 wherein said formulation further comprises one or more excipients.

43. The method of claim 42 wherein said one or more excipients comprise a cryoprotectant, a tonicity modifier, a bulking agent or any combination thereof.

44. A method of treating or preventing a disorder in a mammal, comprising administering to said mammal a pharmaceutically effective amount of a pharmaceutical formulation prepared by the method of claim 31.

45. The method of claim 44 wherein said ansamycin is selected from the group consisting of:

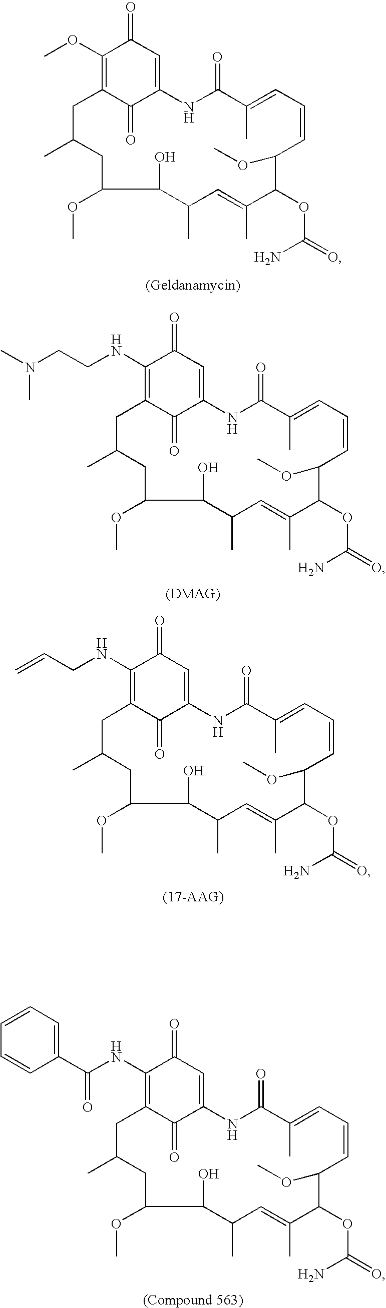

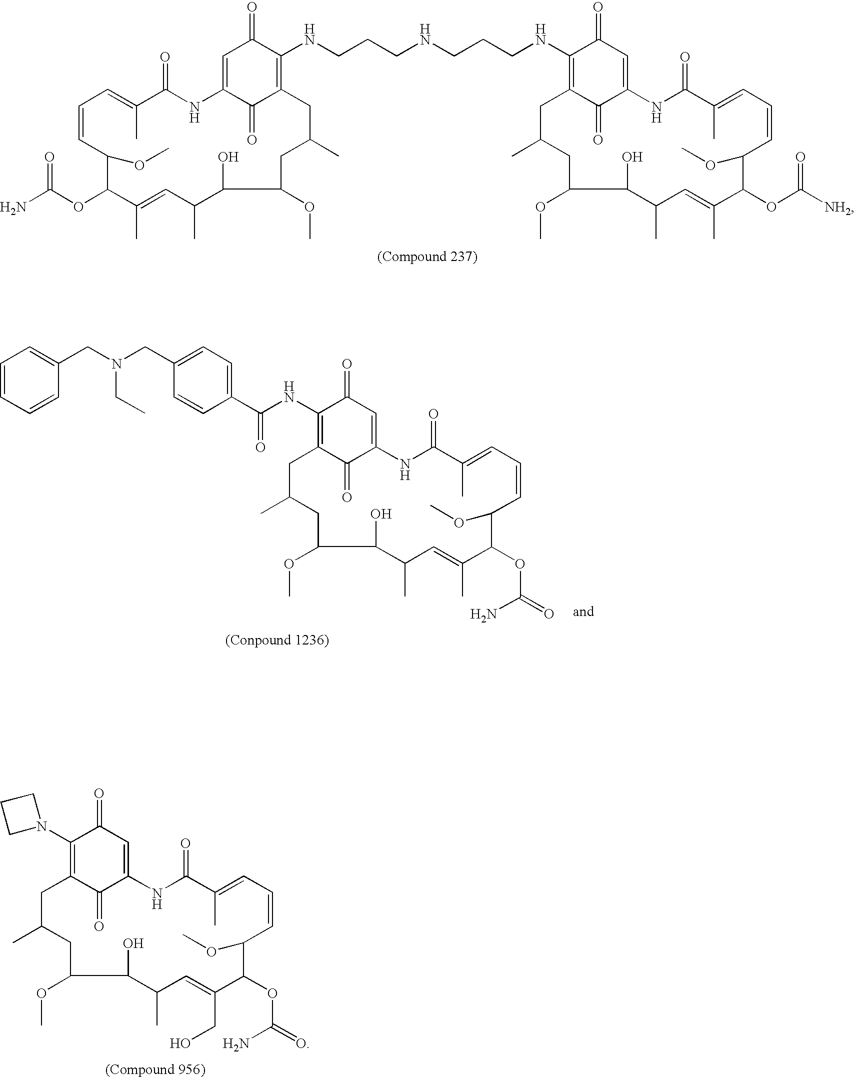

46. The method of claim 44 wherein said ansamycin is 17-AAG.
